(19) United States
(12) Patent Application Publication
(10) Pub. No.: US 2023/0144427 A1
(43) Pub. Date: May 11, 2023

(54) COMPOSITION AND METHODS FOR IMPROVING THICKNESS AND RECEPTIVITY OF ENDOMETRIAL LINING

(71) Applicants: Vasanthi PALANIVEL, Bangalore (IN); Shrinivas RANGACHARI, Chennai (IN)

(72) Inventors: Vasanthi PALANIVEL, Bangalore (IN); Shrinivas RANGACHARI, Chennai (IN)

(21) Appl. No.: 17/626,661

(22) PCT Filed: Jul. 13, 2020
(86) PCT No.: PCT/IB2020/056576

§ 371 Date: Jan. 12, 2022

(30) Foreign Application Priority Data
Jul. 12, 2019 (IN) 201941027999

(51) Int. Cl. A61K 38/18; A61K 47/10; A61K 45/06

(52) U.S. Cl. CPC A61K 38/18; A61K 47/10; A61K 45/06

(57) ABSTRACT

The present disclosure provides compositions and methods for managing female infertility, caused by reduced thickness and receptivity of the endometrial lining. More particularly, the present disclosure provides a platelet derived growth factor concentrate and a composition comprising the same, preferably in combination with a stimulus responsive polymer. Consequently, methods to obtain the said compositions, along with therapeutic applications in improvement in thickness and receptivity of the endometrial lining are provided.

### CROSS-REFERENCE TO RELATED APPLICATIONS

This patent application claims priority to Indian provisional patent application no. 201941027999 filed on Jul. 12, 2019, the complete disclosures of which, in their entireties, are hereby incorporated by reference.

### TECHNICAL FIELD

The present disclosure generally relates to the field of infertility, and in particular female infertility. Accordingly, the present disclosure provides for compositions and methods for managing female infertility, caused by reduced thickness and receptivity of the endometrial lining More particularly, the present disclosure provides a platelet derived growth factor concentrate and a composition comprising the same, preferably in combination with a stimulus responsive polymer. Consequently, methods to obtain the said compositions, along with therapeutic applications in improvement in thickness and receptivity of the endometrial lining are provided.

### BACKGROUND OF THE INVENTION

Infertility is experienced by one in six couples worldwide at least once during their reproductive lifetime and the current prevalence of infertility lasting for at least 12 months is estimated to be around 9 for women aged 20-44 years. Throughout the world, there is a tremendous increase in number to people resorting to In-vitro fertilization IVF to fulfill their dreams of having a baby. Three decades after the introduction of IVF ongoing pregnancy ratesper cycle vary between 8.6 and 46.2 Centre for Medically Assisted Procreation, 2009 American Society for Reproductive

Medicine, 2011 Human Fertilisation & Embryology Authority, 2011 Nederlandse Vereniging voor Obstetric & Gynaecologie, 2011. Considering these relatively limited success rates versus the associated risks and costs of IVF, knowledge on predictive factors for the occurrence of pregnancy is instrumental.

Along with maternal age, endometrial characteristics, such as endometrial pattern, sub endometrial blood flow and endometrial thickness EMT, have been described as prognostic factors in determining success of IVF. It has been suggested that thin endometrium is associated with lower IVF success rates.

The human uterus mainly consists of the endometrium and the outer smooth muscle layer termed the myometrium. The functional layer of the human endometrium is a highly regenerative tissue undergoing monthly cycles of growth, differentiation and shedding during a woman's reproductive years. Fluctuating levels of circulating estrogen and progesteroneorchestrate this dramatic remodeling of human endometrium. Endometrial regeneration also follows parturition and endometrial resection. Endometrial regeneration from the basal layer contributes to the replacement of the functionalis layer followed by its slough off during menses and parturition. However, the endometrium may fail to respond to estrogen and not regenerate in certain pathologies, for example, Asherman's Syndrome and atrophy of the endometrium. Such subjects may experience abnormal endometrial proliferation and become infertile.

Successful embryo implantation during IVF cycle requires an appropriate embryonic development coincident with a receptive endometrium. In clinical practice, adequate endometrial growth is required for successful implantation. The minimal endometrial thickness required for embryo transfer is 7 mm at the end of follicular phase Khalifa E et al., 1992. Thin endometrium non-responsive to standard treatments is still a challenge in assisted reproductive technique ART, and is widely considered sub-optimal for transfer and associated with reduced pregnancy chances. This requires repeated expensive IVF cycles, cycle cancellations, unplanned cryopreservation of embryos and, in the most extreme cases in the utilization of gestational carriers.

However, a number of women with thin endometrium remain non-responsive with repeated cycles of conventional remedies. There is a need for new and more effective therapies for patients with persistent thin endometrial lining in in vitro fertilization IVF treatment programs, particularly for patients resistant to standard therapies. Regenerative medicine is offering solutions and hope for people who have conditions that today are beyond repair. During past few years a considerable progress in the field of regenerative medicine has been made.

During natural wound healing, activated platelets concentrate in the wound area and secrete a plethora of factors that play an instrumental role in not only coordinating wound healing but also in establishing normal tissue architecture and efficient tissue remodeling. For a physiologically highly regenerative tissue like endometrium, growth factor cocktail accelerating the natural proliferation of endometrial cells and enhancing the tissue remodeling (epithelial, endothelial and stromal layer integrity) to get a good quality trilaminar pattern is crucial. As against other specialities, in ART/IVF procedures, every event is time bound and to avoid cycle cancellation, preparation of endometrium in the current cycle is very crucial which is difficult by single bioactive agent like G-CSF or conventional PRP. To improve endometrial quality, repeated administrations (upto 5 times) within a period of 15-20 days is required. So the subject has to be pricked multiple times to donate blood for PRP preparation. As against other specialties, in ART/IVF procedures, every event is time bound and to avoid cycle cancellation and associated huge financial loss and emotional trauma, preparation of endometrium in the current cycle is very crucial which is difficult by single bioactive agent like G-CSF or conventional PRP.

Extracting therapeutic levels of platelets and subsequently growth factors by appropriate activation method has been a technical challenge requiring technicians to operate the equipment originally designed for the production of platelet rich plasma (PRP). Moreover, conventional PRP includes lymphocytes which are pro-inflammatory in nature which doesn't favor embryo implantation.

In summary, there is a need in the art to develop a combination of autologous blood derived growth factors with various angiogenic and anti-inflammatory proteins to improve endometrial thickness and receptivity in patients undergoing frozen embryo transfer cycles during IVF procedures.

### SUMMARY OF THE DISCLOSURE

In order to remedy the issues in prior art, the present disclosure provides a platelet-derived growth factor concentrate, wherein the platelet-derived growth factor concentrate is substantially free of platelets, RBCs and WBCs.

In some embodiments, the growth factors concentrate comprises growth factor(s) selected froma group comprising VEGF, EGF, bFGF, IGF-1, PDGF-BB, TGF-b1 and combinations thereof.

In some embodiments, concentration of the VEGF ranges from about 500-1300 pg/mL, concentration of the EGF ranges from about 100-2000 pg/mL, concentration of the bFGF ranges from about 25-500 pg/mL, concentration of the IGF-1 ranges from about 500-1000 ng/mL, concentration of the PDGF-BB ranges from about 20-500 ng/mL, and concentration of the TGF-b 1 ranges from about 100-2000 ng/mL.

In some embodiments, the PRP that the GFC is prepared from comprises a platelet count that is about 10 to 20-fold greater than starting whole blood sample from same subject, a red bloodcell (RBC) count that is 60 to 90-fold lower than starting whole blood sample from same subject, and/or a white blood cell (WBC) count that is about 10 to 99-fold lower than starting whole blood sample from same subject

Further provided herein is a therapeutic composition comprising the growth factor concentrate as described above and a thermoresponsive polymer.

In some embodiments, the thermoresponsive polymer is selected from a group comprising a copolymer of poly(N-isopropylacrylamide-co-n-butyl methacrylate) and polyethylene glycol, copolymer comprising poly(ethylene oxide) (PEO) and poly(propylene oxide) (PPO), a NIPAM based polymer, amphiphilic block copolymers, ABA triblock copolymers and poloxamer/pluronics family, and any combination thereof.

In some embodiments, said composition further comprises peripheral blood stem cells (PBSCs)at a concentration ranging from about 10% to 50%.

In further embodiments, said composition further comprises therapeutic agent selected from the group comprising Vitamin E, human chorionic gonadotropin(HCCG),leukemia inhibitory factor (LIF), Vascular endothelial growth factor (VEGF), Metalloproteinase-9 (MMP-9), Aspirin,

Heparin, Sildenafil citrate, estrogen, progesterone, Stem cells, Cells/Stem cell secretome; and wherein the therapeutic composition is fortified with growth factor(s) selectedfrom a group comprising TGF, EGF, 1-IGF-1, bFGF, PDGF, LIF, VEGF, SCF, IL-lb, Fibronectin, IL-1, CSF, HIF-alpha, Activin A, IL-8, TNF-a, NF-kB and any combination thereof.

The present disclosure further provides method for preparing the growth factor concentrate as described above, comprising steps of:
a. incubating whole blood with red blood cell (RBC) aggregating agent(s);
b. subjecting the whole blood incubated with the RBC aggregating agent to a first centrifugation to obtain a supernatant containing platelets;
c. subjecting the supernatant to a second centrifugation to obtain a platelet pellet and platelet-poor plasma (PPP);
d. re-suspending the platelet pellet in PPP to obtain platelet-rich plasma (PRP);
e. treating the PRP with platelet-activation buffer; and
f. collecting supernatant containing the growth factor concentrate.

Further provided herein is a method for preparing the aforementioned therapeutic composition, comprising mixing the platelet derived growth factor concentrate with the thermoresponsive polymer to obtain the composition.

The present disclosure further provides a method for treating an endometrial defect causing infertility in a subject in need thereof comprising, administering to the subject the growth factor concentrate or the therapeutic composition as described above.

The present disclosure further relates to the growth factor concentrate or the therapeutic composition for use in preparing a medicament to improve endometrial thickness and receptivity in IVF procedure.

Further provided herein in a kit for preparing the therapeutic composition of the present disclosure, comprising:
a. RBC activating agent(s) selected from a group comprising: heparin, collagen, a calcium salt, hyaluronic acid, polygeline, thrombin, gelatin, EDTA, sodium citrate, starch, and any combination thereof;
b. a thermoresponsive polymer; and
c. an instruction manual.

Said kit, in further embodiments, further comprises platelet activating agent selected from a group comprising collagen, a calcium salt, hyaluronic acid, thrombin, and any combination thereof, and/or GCSF.

Further optional components of the aforesaid kit include a blood collection container comprising an anti-coagulant, additional therapeutic agent(s) selected from a group comprising Vitamin E, human chorionic gonadotropin(HCCG), leukemia inhibitory factor (LIF), Vascularendothelial growth factor (VEGF), Metalloproteinase-9 (MMP-9), Aspirin, Heparin, Sildenafil citrate, estrogen, progesterone, Stem cells, Cells/Stem cell secretome; and fortifying growth factor(s) selected from a group comprising TGF, EGF, 1-IGF-1, bFGF, PDGF, LIF, VEGF, SCF, IL-1b, Fibronectin, IL-1, CSF, HIF-alpha, Activin A, IL-8, TNF-a, NF-kB and any combination thereof.

The present disclosure further relates to use of the thermoresponsive polymer for preparing a medicament for improving fertility. In an embodiment, the thermoresponsive polymer is selected from a group comprising a copolymer of poly(N-isopropylacrylamide-co-n-butyl methacrylate) and polyethylene glycol, copolymer comprising poly(ethylene oxide) (PEO) and poly(propylene oxide) (PPO), a NIPAM based polymer, amphiphilic block copolymers, ABA triblock copolymers and poloxamer, and any combination thereof.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with detailed description below, are incorporated in and form part of the specification, and serve to further illustrate the embodiments and explain various principles and advantages, where:

FIG. 1 represents chemical formula (A) and representation of volume phase transition (B) between coil (left) and globular (right) hydrogel conformations of a NIPAM based polymer.

FIG. 2 represents (A) the swollen PNIPAAm hydro-sol in aqueous solution below critical temperature (Tc) of 32° C. and (B) the shrunken dehydrated PNIPAAm hydrogel above critical temperature (Tc) of 32° C.

FIG. 3 represents schematic scheme for preparing the composition of the present disclosure and the subsequent administration into the uterus.

FIG. 4 represents impact of inclusion of RBC aggregators in the PRP/GFC protocol.

FIG. 5 represents the effect of the platelet activation step of the present disclosure in terms of the concentration of a) VEGF b) EGF c) bFGF d) IGF-1 e) PDGF-BB f) TGF-b1 in the platelet derived growth factor concentrate.

FIG. 6 represents the in vitro growth factor release kinetics for comparing the composition of the present disclosure with a preparation devoid of the thermoresponsive polymer.

FIG. 7 panels A-H represent the images of various stages of whole blood processing for preparing the PRP and the GFC of the present disclosure. Panel A shows whole blood drawn from a patient and collected into acid citrate dextrose (ACD-A) solution gel tube/K2 EDTA tube. Panel B shows settling of RBCs upon incubation of the whole blood for 45 minutes with a buffer comprising one or more RBC aggregating agents. Panel C shows the whole blood after first centrifugation at 600 rpm for 2 minutes the bottom layer contains RBCs and WBCs and the supernatant contains platelets-containing plasma. Panel D shows the supernatant containing platelets-containing plasma transferred to another centrifugation tube. Panel E shows the platelet pellet obtained after the second centrifugation step at 3000rpm for 10 minutes. Panel F shows the gel-like consistency of PRP during the platelet activation stage. Panels G and H show separation of platelets in the form of a clot-like structure from the supernatant containing the growth factor concentrate.

FIG. 8 depicts a comparison of the RBC and WBC count between the GFC of the present disclosure and the starting whole blood.

FIG. 9 represents the design of the non-randomized study for evaluating the safety and efficacy of GFC isolated from peripheral blood. ‘ABCD’ in said figure refers to the GFC of the present disclosure.

### DETAILED DESCRIPTION

In view of the drawbacks associated, and to remedy the need created by the art available in the field of female infertility, more particularly with regard to endometrial defects such as reduced thickness and receptivity of the endometrial lining, the present disclosure provides a plasma-derived growth factor concentrate (GFC) and compositions comprising the same in combination with a stimulus responsive polymer.

Said GFC of the present disclosure is prepared from Platelet Rich Plasma (PRP). Said PRP that the GFC is obtained from could be conventional PRP, or PRP specifically prepared as per the protocol provided in the present disclosure.

The GFC and compositions having the GFC and a stimulus responsive polymer help in the improvement of thickness and receptivity of the endometrial lining which is particularly relevant in the field of Assisted Reproductive Technology (ART) such as IVF.

While the GFC by itself provides for improvement of thickness and receptivity of the endometrial lining which is crucial for successful embryo implantation, the inclusion of a stimulus responsive polymer, more particularly a thermoresponsive polymer, helps in greater retention of the composition at the site of the administration. Thus, the present disclosure provides for technically advanced compositions for the improvement of thickness and receptivity of the endometrial lining.

However, before describing the compositions of the present disclosure, the corresponding methods and the applications thereof in greater detail, it is important to take note of the common terms and phrases that are employed throughout the instant disclosure for better understanding of the technology provided herein.

Throughout the present disclosure, the term “platelet rich plasma (PRP)” is used to mean conventional PRP or the PRP prepared specifically by the method of the present disclosure. Thus, unless otherwise specifically stated, the general use of the term “platelet rich plasma” or “PRP” throughout the disclosure is understood to interchangeably mean conventional PRP or the PRP prepared by the method of the present disclosure. The PRP prepared by the method of the present disclosure is also referred to herein as the “PRP prepared by the present disclosure” or the “PRP of the present disclosure”. While the method specifically employed to prepare PRP in the present disclosure will be explained in greater detail below, the conventional PRP is any PRP known in the art prepared by previously known methods and technologies, including the buffy coat method. A person skilled in the art is therefore able to refer to the literature and common general knowledge to prepare the conventional PRP quite easily. An example of methods for preparing the conventional PRP is summarized in a review article entitled “Principles and Methods of Preparation of Platelet-Rich Plasma: A Review and Author's Perspective”, J Cutan Aesthet Surg. 2014 October-December; 7(4): 189-197.

Throughout the present disclosure, the terms “growth factor concentrate” or “platelet-derived growth factor concentrate” or “platelet growth factor concentrate” or “GFC” are used interchangeably and refer to a substantially cell-free supernatant comprising a milieu of growth factors, cytokines, and other proteins obtained from lysis of activated platelets from the platelet rich plasma (PRP). As mentioned above, this PRP could be either a conventional PRP or PRP prepared by the present disclosure. The growth factor concentrate (GFC) of the present disclosure is substantially free of cells as upon obtaining of the PRP, the activated platelets are lysed for the said preparation of the growth factor concentrate. The ruptured platelets are then allowed to settle down, and the substantially cell-free supernatant is collected. Preferably, the growth factor concentrate is prepared from the PRP prepared by the present disclosure, which is characterized by high platelet count and very low RBC and WBC count compared to the conventional PRP. As the PRP of the present disclosure has high platelet count and very low levels of RBC and WBC contamination compared to conventional PRP, the growth factor concentrate prepared from the PRP prepared by the present disclosure also has improved characteristics than growth factor concentrates prepared from conventional PRP.

Throughout the present disclosure, the term “stimulus responsive polymer” is used to mean a polymer that is sensitive to or responds to one or more stimuli, which include thermal stimuli, optical stimuli, mechanical stimuli, pH stimuli, chemical stimuli, environmental stimuli or biological stimuli. Preferably, the stimulus responsive polymers employed in the present disclosure are polymers that are sensitive or responsive to thermal stimuli. Accordingly, the stimulus responsive polymer is preferably used to mean a thermoresponsive polymer in the context of the present disclosure. These polymers are temperature-responsive polymers that exhibit a drastic and discontinuous change of their physical properties with change in temperature. For example, these polymers could be in liquid form at certain temperatures, and have the ability of quickly converting into a gel form at increased temperatures.

Throughout the present disclosure, since each of the compositions provide for a therapeutic effect in treatment of female infertility caused due reduced thickness and receptivity of the endometrial lining, the term “composition” is also meant to be understood as “therapeutic composition” and the two are used interchangeably herein.

Throughout the present disclosure, the terms “subject” or “patient” are used interchangeably and refer to a mammal In some embodiments, the subject is a human In some embodiments, the subject is a non-human mammal, including cats, dogs, dairy animals such as cows, sheep, goat, and the like.

Throughout the present disclosure, the abbreviation ‘IVF’ has been used to refer to ‘In vitro fertilization’ and envisages various techniques used in the art to facilitate the IVF protocol. Further, usage of the abbreviation ‘ART’ refers to ‘Assisted Reproductive Technology’. Technical terms used in the Examples in relation to protocols/studies pertaining to said technology are those routinely used in the art. Said terms are within the scope of knowledge of a person skilled in this field and particularly, these technologies.

Abbreviations for commonly used units have been used throughout the disclosure. Said abbreviations are widely used in the art.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” wherever used, will be understood to imply the inclusion of a stated element, integer or step, or group of elements, integers or steps, but not the exclusion of any other element, integer or step, or group of elements, integers or steps. Further, use of the term ‘about’ before values defined in the present disclosure envisages values of about ±10%.

Accordingly, to reiterate, the present disclosure relates to a plasma derived growth factor concentrate (GFC) that is obtained from Platelet Rich Plasma (PRP). Said GFC may be derived from conventional PRP or specifically, PRP of the present disclosure. Exemplary growth factors present in the growth factor concentrate (GFC) of the present disclosure include, but are not limited to, platelet-derived growth factor (PDGF), transforming growth factor (TGF), platelet-derived angiogenesis factor (PDAF), vascular endothelial growth factor (VEGF), epidermal growth factor (EGF), insulin-like growth factor (IGF), basic fibroblast growth factor (bFGF), stromal cell derived factor 1 (SDF-1), hepatocyte growth factor (HGF) and any combination thereof.

In some embodiments of the present disclosure, the growth factor concentrate comprises growth factor(s) selected from a group comprising VEGF, EGF, bFGF, IGF-1, PDGF-BB and TGF-bl or any combination thereof.

As the term suggests, the GFC is a concentrated form of growth factors that are originally present in the platelets. Upon platelet-activating treatment, the activated platelets release the said growth factors in the plasma. Accordingly, the concentration of the growth factors in the GFC is about 4 to 10-fold, about 4 to 8-fold, about 5 to 10-fold, about 5 to 8-fold, about 6 to 10-fold, or about 6 to 8-fold, including values and ranges therebetween, higher than that of the starting whole blood sample.

Exemplary levels of certain growth factors in the growth factor concentrate of the present disclosure are shown in the Table below:

TABLE 1

| | | |
|---|---|---|
| | Concentration range in | |
| | the freshly-prepared | Concentration range in |
| | GFC derived from | the freshly- |
| Growth Factor | conventional PRP | prepared ABCD |

| | | | | |
|---|---|---|---|---|
| VEGF | 500-800 | pg/mL | 500-1300 | pg/mL |
| EGF | 100-200 | pg/mL | 100-2000 | pg/mL |
| bFGF | 25-75 | pg/mL | 25-500 | pg/mL |
| IGF-1 | 70-130 | ng/mL | 500-1000 | ng/mL |
| PDGF-BB | 20-85 | ng/mL | 20-500 | ng/mL |
| TGF-β1 | 250-350 | ng/mL | 250-2000 | ng/mL |

In some embodiments, the GFC may have one or more of the above reported enhanced levels of growth factors.

While the GFC can be prepared from conventional PRP, in some embodiments, it is preferred that the GFC is obtained from the PRP of the present disclosure. Said PRP of the present disclosure, in some embodiments, is characterized by a platelet count that is about 10 to 20-fold greater than starting whole blood sample from same subject; a red blood cell (RBC) countthat is 60 to 90-fold lower than starting whole blood sample from same subject, and/or a whiteblood cell (WBC) count that is about 10 to 99-fold lower than starting whole blood sample from same subject.

In some embodiments, said PRP prepared by the protocol described in later paragraphs of the present disclosure comprises about 10, 11, 12, 13, 14, or 15-fold more platelets, including values and ranges therebetween, compared to the starting whole blood sample from which the PRP is prepared. In some embodiments, the PRP of the present disclosure comprises about 10 to 12-fold, 10 to 13-fold, 11 to 14-fold, 12 to 14-fold, 12 to 15-fold more platelets, including values and ranges therebetween, compared to the starting whole blood sample. In an exemplary embodiment, if the starting whole blood sample of a subject comprises about 150×10³ platelets per microliter, the PRP prepared according to the present disclosure can comprise about 2040 platelets per microliter, which is about 13.6-fold greater than the starting whole blood sample. In another exemplary embodiment, for a whole blood sample of a subject comprising about 230×10³ platelets per microliter, the PRP of the present disclosure comprises platelets in the range of about 2300 to 3450×10³ per microliter, which is about 10 to 15-fold greater than the starting whole blood sample.

The PRP of the present disclosure is preferably autologous. However, allogenic PRP and use of allogenic PRP is also contemplated. In some embodiments, the PRP is prepared from venous blood. In some embodiments, the PRP is prepared from cord blood or bone marrow. It is known in the art that platelets serve as a reservoir of growth factors, cytokines, and other proteins. These growth factors, cytokines, and several other proteins are contained in the alpha-granules of platelets and are released upon activation of platelets.

Said GFC of the present disclosure finds application, independently or in combination with pharmaceutically acceptable excipients in treatment modules to increase thickness of endometrial lining and receptivity. While all pharmaceutically acceptable excipients may be employed in combination with the GFC for the purposes to delivery to the desired site, of particular interest is a combination of the GFC with a stimulus responsive polymer.

Accordingly, the present disclosure relates to a composition comprising the afore-described GFC and a stimulus responsive polymer. In preferred embodiments, the stimulus responsive polymer is a thermoresponsive polymer.

Although not limited in application, in preferred embodiments, the said composition of the present disclosure find application in the enhancement of the thickness and receptivity of the endometrial lining as part of infertility treatment protocols such as IVF.

The present disclosure therefore provides a therapeutic composition having the GFC and the stimulus responsive polymer, wherein the GFC comprises growth factor(s) selected from a group comprising VEGF, EGF, bFGF, IGF-1, PDGF-BB and TGF-b1 or any combination thereof.

The GFC is prepared by subjecting the activated platelets in the PRP to one or more platelet-activating treatments. These are described in further details in the later paragraphs of the present disclosure.

In the therapeutic composition of the present disclosure comprising the GFC and the thermoresponsive polymer, concentration of the VEGF ranges from about 500-1300 pg/mL, concentration of the EGF ranges from about 100-2000 pg/mL, concentration of the bFGF ranges from about 25-500 pg/mL, concentration of the IGF-1 ranges from about 500-1000 ng/mL, concentration of the PDGF-BB ranges from about 20-500 ng/mL, and concentration of the TGF-b1 ranges from about 250-2000 ng/mL.

In some embodiments, in addition to growth factors from autologous blood, therapeutic compositions are further fortified with exogenously added growth factors to provide a concentration of growth factors that is about 4 to 10 times higher than the baseline concentration of corresponding growth factors in starting whole blood. Accordingly, in some embodiments, in the therapeutic compositions, concentration of the VEGF ranges from about 500 to 3000 pg/mL, concentration of the EGF ranges from about 100 to 3000 pg/mL, concentration of the bFGF ranges from about 25 to 3000 pg/mL, concentration of the IGF-1 ranges from about 500 to 3000 ng/mL, concentration of the PDGF-BB ranges from about 20 to 3000 ng/mL, and concentration of the TGF-β1 ranges from about 100 to 3000 ng/mL.

In some embodiments, the stimulus responsive polymer is a thermoresponsive polymer that is selected from a group comprising a copolymer of poly(N-isopropylacrylamide-co-n-butyl methacrylate) and polyethylene glycol, copolymer comprising poly(ethylene oxide) (PEO) and poly(propylene oxide) (PPO), a NIPAM based polymer, amphiphilic block copolymers, ABA triblock copolymers and poloxamer or pluronics family, and any combination thereof. A particularly preferred choice of the thermoresponsive polymer is a copolymer of poly(N-isopropylacrylamide-co-n-butyl methacrylate) and polyethylene glycol.

Though the concentration of components in the above-described composition may vary based on the blood profile of the subject that the blood is initially withdrawn from and the subject to whom the composition is administered, in exemplary embodiments, the therapeutic composition of the present disclosure, concentration of the growth factor(s) in the composition ranges from about 10% to 90%. Further, in some embodiments, concentration of the stimulus responsive polymer in the above described composition ranges from about 10% to 50%.

Accordingly, within the ambit of the present disclosure, the concentration of the thermoresponsive polymer can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%.

Also, Accordingly, within the ambit of the present disclosure, the concentration of the GFC can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%.

In some embodiments, the GFC and the thermoresponsive polymer are present in the composition of the present disclosure at a volume/volume ratio of 90:10 to 10:90.

In some embodiments, the GFC and the thermoresponsive polymer are present in the composition of the present disclosure at a volume/volume ratio of 90:10 to 50:50.

An advantage of the afore-described composition is that it is substantially cell-free. Of particular relevance and importance is the substantial absence of RBCs and WBCs from the above mentioned therapeutic composition of the present disclosure, wherein said cells are known to induce a pro-inflammatory effect.

In some embodiments of the present disclosure, apart from the GFC and the thermoresponsive polymer, the composition of the present disclosure may also comprise peripheral blood stem cells (PBSCs) or endothelial progenitor cells. These PBSCs are a direct result of Endogenous Stem Cell Mobilisation (ESCM) done prior to preparing of the composition. Combining the compositions with PBSCs proves to be more effective as it ensures local availability of the composition for a longer time thereby ensuring improved sperm maturation and vitality. Accordingly, in some embodiments, the therapeutic compositions of the present disclosure comprise PBSCs in addition to the PRP or the growth factor concentrate, along with the thermoresponsive polymer.

In some embodiments, concentration of the PBSCs or the endothelial progenitor cells within the therapeutic composition of the present disclosure ranges from about 10% to 50%. It is important to note that the compositions of the present disclosure comprise GFC, which is derived from whole blood of a subject. Accordingly, as is well known and understood by a person skilled in the art, the internal composition of the whole blood, including the number of cells, proteins, active agents, growth factors etc. varies from subject to subject. Therefore, the GFC so prepared varies accordingly, and so do the additional elements, including the PBSCs, and thus arises a need for a range of concentrations within which the compositions of the present disclosure can be prepared and applied. Accordingly, within the ambit of the present disclosure, the concentration of the PBSCs can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%,48%, 49% or 50%.

Throughout this disclosure, if the concentration of PRP/GFC is expressed in terms of percentages, it refer to the volume of PRP/GFC added to the composition e.g., 30% PRP/GFC means 300 μl of PRP/GFC is added to make 1 ml of the composition or 3 ml of PRP/GFC is added to make 10 ml of the composition.

Throughout this disclosure, if the concentration of PBSCs is expressed in terms of percentages, it refer to the volume of PBSC solution added to the composition e.g., 40% PBSCs means 4 ml of PBSC solution is added to make 10 ml of the composition.

Now, in order for the PBSCs or the endothelial progenitor cells to be included in the compositions of the present disclosure, Bone-Marrow Derived Stem Cell (BMSC) mobilization is stimulated by Granulocyte-Colony Stimulating Factor (G-CSF) said stimulation, in some embodiments of the present disclosure being performed prior to the withdrawal of blood for the preparation of the GFC of the composition of the present disclosure or composition comprising the same. These cells migrate into affected tissues and contribute to tissue repair. Accordingly, in a non-limiting embodiment, prior to the withdrawal of blood for preparation of the compositions of the present disclosure, the subject is administered with Granulocyte-Colony Stimulating Factor (G-CSF).

G-CSF is a cytokine secreted by various tissues that stimulates the proliferation, differentiation and function of neutrophil precursors and mature neutrophils. G-CSF naturally stimulates BMSC mobilization. Contrary to most tissues in which SDF-1 is secreted consequent to an injury or a degenerative condition, in the bone marrow SDF-1 is constitutively produced and released, and binding of SDF-1 to its exclusive receptor CXCR4 leads to the externalization of adhesion molecules, namely integrins, which allow for the adherence of stem cells to the bone marrow matrix. The binding of SDF-1 to CXCR4 is referred to as the SDF-1/CXCR4 axis. The general understanding is that disruption of the SDF-1/CXCR4 axis reduces the expression of adhesion molecules, leading to a reduction in the adherence of stem cells to the bone marrow matrix and the consequent mobilization of stem cells.

Various compounds known to trigger stem cell mobilization all affect the SDF-1/CXCR4 axis in various ways. For example, G-CSF disrupts the SDF-1/CXCR4 axis by activating a series of proteolytic enzymes including elastase, cathepsin G, and various matrix metalloproteinases (MMP2 and MMP9) that inactivate SDF-1 (Mannello et al., 2006; Jin et al., 2006; Cation et al., 2003).

In some embodiments, administration of G-CSF enhances the concentration of WBCs in the blood by about 5-folds, when compared to whole blood analyzed without stimulation by G-CSF.

Without wishing to be bound by any theory, it is understood that the naturally mobilized BMSC can traffic to various areas of the body and contribute to tissue regeneration and repair through peripheral blood as peripheral blood stem cells (PBSCs). As PBSCs play a key role in the process of SC-mediated tissue repair, employing PBSCs in a tissue regenerative composition like the ones of the present disclosure constitutes a therapeutic approach. In view of said rationale, in an embodiment of the present disclosure, a portion of the withdrawn blood is employed to isolate PBSCs, which are then included as part of the compositions of the present disclosure.

In exemplary embodiments, said isolated PBSCs are added to the platelet derived growth factor concentrate (GFC) for therapeutic applications. The aspect of isolation of PBSCs and their combination with the platelet derived growth factor concentrate of the present disclosure is performed by methods generally known in the art. This is further elaborated on in further sections of the present disclosure.

Thus, in some embodiments, the present disclosure provides compositions that comprise thermosensitive polymer; GFC obtained from conventional PRP or PRP of the present disclosure; and optionally, peripheral blood stem cells (PBSCs).

In some embodiments, said composition comprises thermosensitive polymer and GFC obtained from conventional PRP. In some embodiments, said composition comprises thermosensitive polymer, GFC obtained from conventional PRP and peripheral blood stem cells (PBSCs). In some embodiments, the composition comprises thermosensitive polymer and GFC obtained from PRP of the present disclosure. In other embodiments, the composition comprises thermosensitive polymer, GFC obtained from PRP of the present disclosure and optionally, peripheral blood stem cells (PBSCs).

In some embodiments of the present disclosure, the compositions described above also comprise one or more additional therapeutic agent selected from a group comprising hormone, growth factor, protein, cell, cell secretome, and drug, or any combination thereof. For example, the composition can comprise any one or more of Vitamin E, human chorionic gonadotropin(HCCG), leukemia inhibitory factor (LIF), Vascular endothelial growth factor (VEGF), Metalloproteinase-9 (MMP-9), Aspirin, Heparin, Sildenafil citrate, estrogen, progesterone, stem cells and cells/stem cell secretome.

The stem cells may include adult or embryonic stem cells and from varied sources including those from bone marrow, adipose tissue, blood, umbilical cord and embryo. Further, any drug that is a therapeutic agent known to a person skilled in the art for the treatment of endometrial defects such as thinning or non-receptivity of the endometrial wall, and which can be employed without any compatibility challenges with the compositions of the present disclosure, are also contemplated within the ambit of the present disclosure.

In some embodiments, the growth factors that are included as additional therapeutic agents in the compositions of the present disclosure include but are not limited to Transforming growth factor (TGF), Epidermal growth factor (EGF), Insulin-like growth factor-1 (IGF-1), Basic fibroblast growth factor (bFGF), Platelet-derived growth factor (PDGF), Leukemia inhibitory factor (LIF), Vascular endothelial growth factor (VEGF), Stem cell factor (SCF), Interleukin 1 beta (IL-1b), Fibronectin, IL-1, colony-stimulating factor (CSF), Hypoxia-inducible factor 1-alpha (HIF-alpha), Activin A, Interleukin 8 (IL-8), Tumour Necrosis Factor alpha (TNF-a) and Nuclear Factor kappa-light-chain-enhancer of activated B cells (NF-kB). In some embodiments, these growth factors help in fortification of the final composition and help in enhancing the overall concentration of the growth factors in the final preparation. As the compositions of present disclosure comprise GFC, which are derived from whole blood of a subject, it is well known and understood by a person skilled in the art that the internal composition of the whole blood, including the number of growth factors vary from subject to subject. Accordingly, the growth factors as part of the additional therapeutic agents help in maintaining the overall levels of growth factors in the final composition.

In some embodiments, when the additional therapeutic agent is a hormone, protein, cell, cell secretome, or drug, or any combination thereof, they are present in the composition at a concentration ranging from about 10% to 50%. Accordingly, within the ambit of the present disclosure, the concentration of the additional therapeutic agent in the composition can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In exemplary embodiments, when the additional therapeutic agent is a hormone, protein, cell, cell secretome, or drug, or any combination thereof, they are present in the composition at a concentration ranging from about 20% to 30%. However, when the additional therapeutic agent is a growth factor, it is present at a concentration which is about 4-fold to 10-fold higher than the constituting whole blood used to prepare the compositions. According, within the ambit of the present disclosure, the concentration of the growth factor in the composition can be any of 4-fold, 5-fold, 6-fold,7-fold, 8-fold, 9-fold or 10-fold higher than the blood starting from which the GFC is prepared.

Thus, in some embodiments, the present disclosure provides compositions that comprise thermosenstive polymer; GFC obtained from either conventional PRP or PRP prepared by the present disclosure; peripheral blood stem cells (PBSCs), and one or more additional therapeutic agent. While the present disclosure provides for compositions as captured in this or the previous embodiments, it is important to note that the present disclosure equally contemplates all other possible permutations-combinations that may be possible from the present disclosure, as long as the compositions at minimum comprise GFC obtained from either conventional PRP or PRP prepared by the present disclosure. Thus, all compositions that comprise the GFC obtained from either conventional PRP or the PRP of the present disclosure and the thermosenstive polymer with both peripheral blood stem cells (PBSCs), and one or more additional therapeutic agent, or comprise only PBSCs without any additional therapeutic agentor comprise only one or more additional therapeutic without any PBSCs are within the ambit of the present disclosure. Also within the ambit of the present disclosure are compositions comprising the GFC obtained from either conventional PRP or the PRP of the present disclosure in combination with both peripheral blood stem cells (PBSCs), and one or more additional therapeutic agent or with only PBSCs and without any additional therapeutic agentor only one or more additional therapeutic and without any PBSCs.

In some embodiments, while the GFC forms the active center of the compositions, it is the thermosensitive polymer that enhances the therapeutic effect by ensuring that the composition is retained by the body at the site of administration for a longer period of time. Since the polymer is thermosensitive in nature, one of the most important properties that it showcases is the conversion of its physical form from liquid to gel, when in contact with physiological temperature. Gelling may be observed at a temperature ranging from about 27° C. to 37° C.Thus, in some embodiments, while it is viscous but in the form of an injectable liquid at room temperature, it transitions to a temporary self-forming polymeric plug at body temperature. For example, the thermoresponsive polymer exists in a liquid form at a temperature ranging from about −20° C. to +27° C., and in a gel form at a temperature ranging from about +27.1° C. to +60° C. Because the material undergoes a temperature-induced phase change with no alteration in the product's chemical composition, it works well to enhance the overall impact of the composition. The use of thermoresponsive polymers in the present disclosure therefore allows for sustained and targeted effect of the therapeutic composition of the present disclosure and prevents leakage from the site of administration or dilution by other bodily fluids.

In some embodiments, the thermoresponsive polymer employed to prepare the compositions of the present disclosure is a synthesized biocompatible polymer, which has no biological contaminants. An example of such a polymer is N-isopropylacrylamide (NIPAM) based polymer, for instance poly(Nisopropylacrylamide-co-n-butyl methacrylate) poly(NIPAAm-co-BMA). The present disclosure therefore provides for compositions that comprise a NIPAM based polymer; GFC obtained from either of conventional PRP or PRP of the present disclosure; optionally along with peripheral blood stem cells (PBSCs), and one or more additional therapeutic agent.

In some embodiments, a thermoresponsive polymer employed to prepare the compositions of the present disclosure includes copolymers composed of thermoresponsive polymer blocks and hydrophilic polymer blocks and is characterized by its temperature-dependent dynamic viscoelastic properties. The thermoresponsive polymer blocks are hydrophilic at temperatures below the sol-gel transition temperature and are hydrophobic at temperatures above the sol-gel transition temperature. The hydrophobic interaction results in formation of a homogenous three-dimensional polymer network in water. In some embodiments, the thermoresponsive polymer block which are part of such copolymers is a NIPAM based polymer. An example ofsuch thermoresponsive polymer blocks is poly(Nisopropylacrylamide-co-n-butyl methacrylate) poly(NIPAAm-co-BMA), which are combined with hydrophilic polymer blocks, including polyethylene glycol (PEG). The present disclosure therefore provides for compositions that comprise a copolymer of poly(Nisopropylacrylamide-co-n-butylmethacrylate) poly(NIPAAm-co-BMA) and polyethylene glycol (PEG); GFC obtained from either conventional PRP or PRP of the present disclosure; optionally along with peripheral blood stem cells (PBSCs), and one or more additional therapeutic agent. As alternatives to PEG, the thermoresponsive polymers can also comprise poly(D,L-lactide-co-glycolide) (PLGA), poly(lactic acid) (PLA), poly(glutamic acid) (PGA), poly(caprolactone) (PCL), N-(2-hydroxypropyl)-methacrylate (HPMA) copolymers, and poly(amino acids). In some embodiments, PEGylated NIPAM polymers can be prepared as described by the methods known in the art.

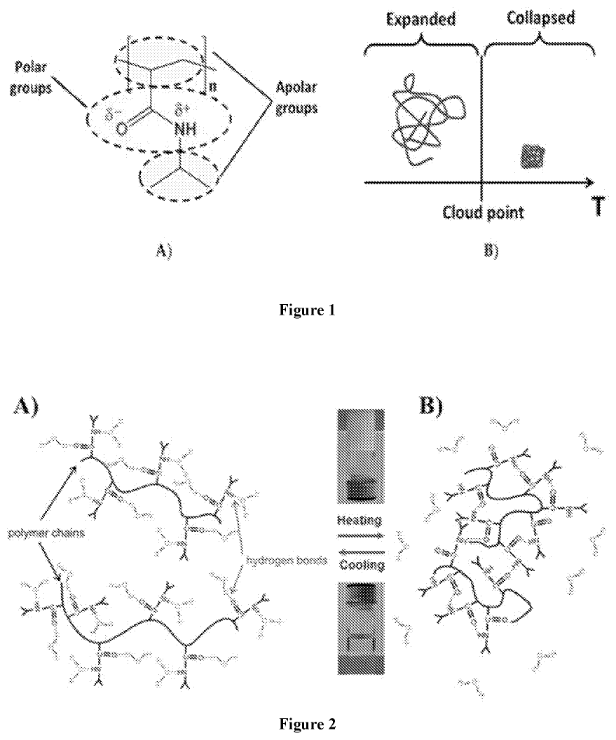

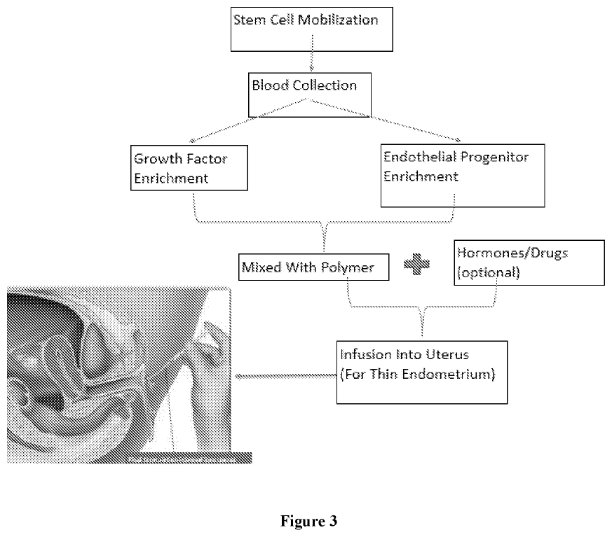

In some embodiments, chemical formula (A) and representation of volume phase transition (B) between coil (left) and globular (right) hydrogel conformations of a NIPAM based polymer is provided by FIG. 1. Further, representation of (A) the swollen PNIPAAm hydro-sol in aqueous solution below critical temperature (T_{c}) of 32° C. and (B) the shrunken dehydrated PNIPAAm hydrogel above critical temperature (T_{c}) of 32° C. is provided by FIG. 2.

In some embodiments, the thermoresponsive polymer employed to prepare the compositions of the present disclosure include amphiphilic block copolymers, or ABA triblock copolymers including poloxamers, such as poloxamer 407. These polymers are biocompatible, highly water-soluble and polymorphic materials, and thus ideal for us in thermosensitive biological applications. While they dissolve conveniently in blood, they are also excreted easily in urine.

In some embodiments, the amphiphilic copolymers include those with hydrophilic block hydrophobic block polymers. An example of such an amphiphilic polymer is a copolymer of poly(ethylene oxide) (PEO) and poly(propylene oxide) (PPO). A commercially available example of such a polymer is Pluronic®.

In some embodiments, a thermoresponsive polymer employed to prepare the compositions of the present disclosure includes any polymer known to a person skilled in the art that possesses thermoresponsive properties. The present disclosure accordingly also contemplates all thermoresponsive polymers that are known in the art, commercially available and/or those approved for medical/therapeutic applications by the U.S. Food and Drug Administration (FDA).

In some embodiments, while the presence of the thermo-responsive copolymer is a mandatory feature of the compositions of the present disclosure, the concentration at which the polymer may be present within the composition can vary over a range depending on the final constituents of the composition, including GFC, PBSCs and/or additional therapeutic agents. Similarly, the concentration of the GFC within the composition also varies over a specified range.

Thus, in some embodiments, concentration of the thermoresponsive polymer within the therapeutic composition of the present disclosure ranges from about 10% to 90%, whereas concentration of the GFC within the therapeutic composition of the present disclosure ranges from about 10% to 90%.

Accordingly, the GFC and the thermoresponsive polymer are present in the compositions of the present disclosure at a ratio ranging from about 90:10 to 10:90.

Since the ratio of the GFC and the thermoresponsive polymer varies from composition to composition depending on the initial constituents of the GFC, and the final application, the present disclosure contemplates all such compositions that satisfy the concentration and ratio requirements set out above. It is important to note that the compositions of the present disclosure comprise GFC, which are derived from whole blood of a subject. Accordingly, as is well known and understood by a person skilled in the art, the internal composition of the whole blood, including the number of cells, proteins, active agents, growth factors etc. varies from subject to subject. Therefore, the GFC so prepared varies accordingly, and thus arises a need for a range of concentrations within which the compositions of the present disclosure can be prepared and applied. Accordingly, within the ambit of the present disclosure, the concentration of the thermoresponsive polymer can be any of 10%, 11%, 12%, 13%, 14%, 15%, 16%, 17%, 18%, 19%, 20%, 21%, 22%, 23%, 24%, 25%, 26%, 27%, 28%, 29%, 30%,31%, 32%, 33%, 34%, 35%, 36%, 37%, 38%, 39%, 40%, 41%, 42%, 43%, 44%, 45%, 46%,47%, 48%, 49% or 50%. Also, Accordingly, within the ambit of the present disclosure, the concentration of the GFC can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%

The present disclosure therefore provides for compositions that comprise a thermoresponsive polymer at a concentration ranging from about 10% to 50%; the GFC obtained from either conventional PRP or PRP of the present disclosure at a concentration ranging from about 10% to 90%; optionally along with peripheral blood stem cells (PB SCs) at a concentration ranging from about 10% to 50%, and one or more additional therapeutic agents at a concentration ranging from about 10% to 50%, preferably 20% to 30%. For example, a composition herein can comprise a thermoresponsive polymer at a concentration of about 20%; GFC obtained from either conventional PRP or PRP of the present disclosure at a concentration of about 30%; along with peripheral blood stem cells (PBSCs) or the endothelial progenitor cells at a concentration of about 50%.

Now in order for the composition of the present disclosure to be manufactured, the present disclosure also provides a method for preparing the GFC of the present disclosure.

Since preferred embodiments of the present disclosure relate to the GFC obtained from PRP of the present disclosure, said PRP being prepared by a specific protocol, the method initially entails preparing the PRP of the present disclosure.

Accordingly, the present disclosure also relates to a method for preparing a PRP, wherein the

PRP comprises a platelet count that is about 10 to 20-fold greater than starting whole blood sample, or a RBC count that is about 60 to 90-fold lower than starting whole blood sample, and/or a WBC count that is about 10 to 99-fold lower than starting whole blood sample. The method broadly comprises treating a whole blood sample with one or more RBC aggregating agents, spinning the blood to sediment RBCs and WBCs, spinning the supernatant to sediment platelets, and re-suspending the platelets in platelet-poor plasma to provide the PRP.

In one embodiment, the method for preparing PRP comprises: (a) incubating a whole blood sample collected in an anti-coagulant container with RBC aggregating agent(s); (b) subjecting the whole blood sample incubated with the RBC aggregating agent to a first centrifugation step to obtain a supernatant containing platelets; (c) subjecting the supernatant to a second centrifugation step to obtain a platelet pellet and platelet-poor plasma (PPP); and (d) re-suspending the platelet pellet in PPP to obtain the PRP.

The above described order of steps is not binding on the method of the present disclosure and does not restrict the order in which the steps must be performed. The steps may be performed in any order that is logically feasible, and known to a person skilled in the art.

In some embodiments, the RBC aggregating agent is selected from a group comprising heparin, collagen, a calcium salt, hyaluronic acid, polygeline, thrombin, gelatin, EDTA, sodium citrate, starch, and any combination thereof. In another exemplary embodiment, the RBC aggregating agent is any one of the above or is a combination of two or more of the above referred aggregating agents. In an exemplary embodiment, the RBC aggregating agent is a combination of heparin, collagen, and a calcium salt. In another exemplary embodiment, the RBC aggregating agent is a combination of heparin, collagen, and a calcium salt hyaluronic acid, polygeline, thrombin. In another exemplary embodiment, the RBC aggregating agent is a combination of polygeline, thrombin, and gelatin. In another exemplary embodiment, the RBC aggregating agent is a combination of thrombin, gelatin, and sodium citrate. In another exemplary embodiment, the RBC aggregating agent is a combination of heparin, polygeline, and starch. In another exemplary embodiment, the RBC aggregating agent is a combination of polygeline, gelatin, and starch. In some embodiments, the RBC activating agent is suspended in a physiologically acceptable buffer. In some embodiments, the RBC aggregating agent is added to the whole blood at a concentration of about 0.2% to 10%, for example, about 0.2%, 0.4%, 0.6%, 0.8%, 1.0%, 2%, 3%, 4%, 5%, 6%, 7%, 8%, 9%, or 10% by volume of the whole blood sample. The whole blood sample is incubated with the RBC activating agent for about 5 minutes to 45 minutes at an ambient temperature. The ambient temperature for incubation ranges from about 4° C. to 37° C., about 10° C. to about 20° C., about 20° C. to 30° C., or about 20° C. to about 25° C. The time of incubation ranges from 5 to 45 minutes, including values and ranges therebetween, such as about 15 minutes, about 20 minutes, about 25 minutes, about 30 minutes, about 35 minutes, about 40 minutes, about 45 minutes, about 15 to 45 minutes, about 30 to 45 minutes, about 10 to 40 minutes, or about 20 to 40 minutes. During the incubation, RBCs aggregate and start settling down.

In some embodiments, the Ca salt is calcium chloride or calcium gluconate or other clinically.

After incubation with the RBC aggregating agent, the whole blood sample is centrifuged (first centrifugation) at a low speed such as about 300 rpm to1000 rpm for about 2 minutes to 10 minutes. In some embodiments, the first centrifugation step is carried out at about 300 to 1000 rpm, about 350 to 950 rpm, about 350 to 800 rpm, about 400 to 900 rpm, about 450 to 950 rpm, about 400 to 800 rpm, about 500 to 1000 rpm, about 500 to 900 rpm, about 500 to 850 rpm, about 500 to 800 rpm, about 550 to 750 rpm, about 550 to 700 rpm, about 550 to 800 rpm, about 600 to 800 rpm, about 650 to 800 rpm, or about 650 to 750 rpm, including values and ranges therebetween. Time for the first centrifugation step ranges from about 2 minutes to 10 minutes, about 2 minutes to 8 minutes, about 2 minutes to 6 minutes, about 2 minutes to 5 minutes, about 2 minutes to 4 minutes, about 2 to 3 minutes, about 3 minutes to 9 minutes, about 3 minutes to 8 minutes, about 3 minutes to 5 minutes, about 3 minutes to 4 minutes, about 4 minutes to 8 minutes, about 5 minutes to 10 minutes, including values and ranges therebetween. The first centrifugation step can be carried out at any of the speed values for any of the time periods described herein. In the first centrifugation step, RBCs and WBCs sediment and platelets remain in the supernatant. Treatment with RBC aggregating agents prior to the first centrifugation ensures efficient removal of RBCs from the whole blood by way of sedimentation.

After the first centrifugation step, the supernatant containing platelets is further centrifuged (second centrifugation step) to sediment platelets. The second centrifugation step is carried out at about 900 to 5000 rpm for about 5 minutes to15 minutes. In some embodiments, the second centrifugation step is carried out at about 900 to 4000 rpm about 5-15 minutes. In some embodiments, the second centrifugation step is carried out at about 1200 to 5000 rpm, 1200 to 4500 rpm,1200 rpm to 3500 rpm, about 1200 rpm to 3200 rpm, about 1400 rpm to 3500 rpm, about 1400 rpm to 3200 rpm, about 1500 rpm to 3500 rpm, about 1500 rpm to 3200 rpm, about 1500 rpm to 3000 rpm, about 1800 rpm to 3500 rpm, about 1800 rpm to 3200 rpm, about 1800 rpm to 3000 rpm, about 2000 rpm to 3000 rpm, about 2200 rpm to 3200 rpm, about 2500 rpm to about 3200 rpm, about 2500 rpm to 3000 rpm, about 2800 rpm to 3200 rpm, about 2900 rpm to 3100 rpm, including values and ranges therebetween for about 5 minutes to 15 minutes, about 5 minutes to 12 minutes, about 5 minutes to 10 minutes, about 6 minutes to 12 minutes, about 6 minutes to 10 minutes, about 8 minutes to 15 minutes, about 8 minutes to 12 minutes, about 10 minutes to 15 minutes, about 10 minutes to 12 minutes, or about 12 minutes to 15 minutes, including values and ranges therebetween. After the second centrifugation step, platelets form a pellet leaving platelet-poor plasma (PPP) as supernatant. PPP is aspirated anda desired volume of PPP is used to re-suspend the platelet pellet to provide platelet-rich plasma. In some embodiments, platelet pellets obtained from about 30 to 60 ml of starting whole blood sample are re-suspended in about 3 ml to 6 ml of PPP to provide PRP.

In some embodiments, a method for preparing PRP comprises: (a) incubating a whole blood sample collected in an anti-coagulant container with RBC aggregating agent(s) selected from a group comprising heparin, collagen, a calcium salt, hyaluronic acid, polygeline, thrombin, gelatin, EDTA, sodium citrate, starch, and any combination thereof, wherein the incubation is carried out at a temperature of about 20° C. to 25° C.; (b) subjecting the whole blood sample incubated with the RBC aggregating agent to a first centrifugation step to obtain a supernatant containing platelets, wherein the first centrifugation is carried out at about 300 rpm 1000 rpm for about 2 minutes to10 minutes; (c) subjecting the supernatant to a second centrifugation step to obtain a platelet pellet and platelet-poor plasma (PPP), wherein the second centrifugation is carried out at about 900 rpm to 4000 rpm for about 5 minutes to 15 minutes; and (d) re-suspending the platelet pellet in PPP to obtain the PRP. Said method for preparing the PRP described herein provides about 10 to 15-fold enrichment of platelets compared to starting whole blood sample, or about 60 to 80-fold reduction in the RBC count compared to starting whole blood sample, and/or about 10 to 30-fold reduction in WBCs, including values and ranges therebetween, compared to starting whole blood sample from same subject.

Accordingly, the present disclosure relates to PRP so prepared by the method of the present disclosure. As mentioned, in some embodiments, the number of platelets, RBCs, and/or WBCs present in the PRP of the present disclosure are characterized in terms of fold increase or fold decrease compared to the starting whole blood sample or conventional PRPs as the number of platelets, RBCs, and WBCs vary from a subject to subject or even for the same subject over the period of time; accordingly, a fold increase/enrichment (for platelets) and/or a fold decrease/reduction (for RBCs/WBCs) effectively characterize or distinguish the PRP of the present disclosure over starting whole blood sample and/or conventional PRPs.

In some embodiments, the platelet count of the PRP of the present disclosure is about 1.2 to 2.5-fold, including values and ranges therebetween, greater than the platelet count of the conventional PRP. In some embodiments, the platelet count of the PRP of the present disclosure is about 1.2, about 1.3, about 1.4, about 1.5, about 1.6, about 1.7, about 1.8, about 1.9, about 2, about 2.1, about 2.2, about 2.3, about 2.4, or about 2.5-fold, including values and ranges therebetween, greater than the platelet count of the conventional PRP. In some embodiments, the platelet count of the PRP of the present disclosure is about 1.2 to 2.2-fold, about 1.2 to 2-fold, about 1.2 to 1.8-fold, about 1.2 to 1.6-fold, about 1.5 to 2.5-fold, 1.5 to 2.2-fold, about 1.5 to 2-fold, including values and ranges therebetween, greater than the platelet count of the conventional PRP.

In some embodiments, the RBC count of the PRP of the present disclosure is about 60 to 80-fold lower, including values and ranges therebetween, compared to the starting whole blood sample. In some embodiments, the RBC count of the PRP of the present disclosure is about 60 to 75-fold, about 60 to 70-fold, about 65 to 80-fold, about 65 to 70-fold, about 65 to 75-fold, about 70 to 80-fold, or about 75 to 80-fold lower, including values and ranges therebetween, compared to the starting whole blood sample. In some embodiments, the RBC count of the PRP of the present disclosure is about 60, about 65, about 70, about 75, or about 80-fold lower, including values and ranges therebetween, compared to the starting whole blood sample. In an exemplary embodiment, if the starting whole blood sample of a subject comprises about 4.7×10⁶ RBCs per microliter, the PRP prepared according to the present disclosure comprises about 0.06×10⁶ RBCs per microliter, which is about 78.3-fold reduction in RBCs than the starting whole blood sample. In another exemplary embodiment, for a whole blood sample of a subject comprising about 5.5×10⁶ RBCs per microliter, the PRP of the present disclosure comprises RBCs in the range of about 0.09 to 0.068×10⁶ per microliter, which is about 60 to 80-fold lower than the starting whole blood sample.

In some embodiments, the RBC count of the PRP of the present disclosure is about 145 to 155-fold, including values and ranges therebetween, reduced compared to the RBC count of the conventional PRP prepared using a single spin method. In some embodiments, the RBC count of the PRP of the present disclosure is about 145 to 150-fold, including values and ranges therebetween, lower than that of the conventional PRP prepared using the single spin method.

In some embodiments, the RBC count of the PRP of the present disclosure is about 15 to 25-fold, or about 15 to 20-fold, or about 18 to 22-fold, including values and ranges therebetween, lower than the RBC count of the conventional PRP prepared using a double spin method.

In some embodiments, the WBC count of the PRP of the present disclosure is about 10 to 30-fold lower, including values and ranges therebetween, compared to the starting whole blood sample. In some embodiments, the WBC count of the PRP of the present disclosure is about 10 to 30-fold, about 10 to 25-fold, about 10 to 20-fold, about 15 to 30-fold, about 20 to 30-fold, or about 22 to 28-fold lower, including values and ranges therebetween, compared to the starting whole blood sample. In some embodiments, the WBC count of the PRP of the present disclosure is about 10, about 11, about 12, about 13, about 14, about 15, about 16, about 17, about 18, about 19, about 20, about 21, about 22, about 23, about 24, about 25, about 26, about 27, about 28, about 29 or about 30-fold lower, including values and ranges therebetween, compared to the starting whole blood sample. In an exemplary embodiment, if the starting whole blood sample of a subject comprises about 4.5×10³ WBCs per microliter, the PRP prepared according to the present disclosure comprises about 0.19×10³ WBCs per microliter,which is about 23.6-fold reduction in WBCs than the starting whole blood sample. In another exemplary embodiment, for a whole blood sample of a subject comprising about 6.5×10³ WBCs per microliter, the PRP of the present disclosure comprises WBCs in the range of about 0.65 to 0.216×10³ per microliter, which is about 10 to 30-fold lower than the starting whole blood sample.

In some embodiments, the WBC count of the PRP of the present disclosure is about 50 to 70-fold, about 55 to 65-fold, or about 55 to 70-fold, including values and ranges therebetween, reduced compared to the WBC count of the conventional PRP. In some embodiments, the WBC count of the PRP of the present disclosure is about 60, about 61, about 62, about 63, about 64, about 65, about 66, about 67, about 68, about 69, or 7 about 0-fold, or about 60 to 70-fold, including values and ranges therebetween, lower than that of the conventional PRP prepared using the single spin method. In some embodiments, the WBC count of the PRP of the present disclosure is about 55, about 56, about 57, about 58, about 59, about 60, about 61, about 62, about 63, about 64, or about 65-fold, or about 55 to 65-fold, including values and ranges therebetween, lower than the WBC count of the conventional PRP prepared using a double spin method.

In some embodiments, the PRP of the present disclosure comprises about 1500-6750×10³ platelets per microliter, including values and ranges therebetween; about 0.05-0.1×10⁶ RBCs per microliter, including values and ranges therebetween; and/or about 0.1-0.45×10³ WBCs per microliter, including values and ranges therebetween.

In some embodiments, even if the platelet count of the PRP of the present disclosure is marginally higher or closer or may overlap with the platelet count of the conventional PRP; the RBC and/or the WBC count of the PRP of the present disclosure are substantially lower than those of the conventional PRP. In other words, the present PRP has substantially more fold reduction in the RBC count and/or the WBC count than the conventional PRP.

The present disclosure contemplates that the PRP can have any one of the cell counts, fold increase, and fold decrease features described herein, or a combination thereof. For example, in one embodiment, the PRP comprises a platelet count that is about 10 to 20-fold greater, including values and ranges therebetween, than starting whole blood sample. In another exemplary embodiment, the PRP comprises a platelet count that is about 10 to 20-fold greater, including values and ranges therebetween, and a RBC count that is 60 to 90-fold lower, including values and ranges therebetween, than starting whole blood sample. In another exemplary embodiment, the PRP comprises a platelet count that is about 10 to 20-fold greater, including values and ranges therebetween, than starting whole blood sample and a WBC count that is 10 to 99-fold lower, including values and ranges therebetween, than starting whole blood sample from same subject. In another embodiment, the PRP comprises a platelet count that is about 10 to 20-fold greater, including values and ranges therebetween; a RBC count that is 60 to 90-fold lower, including values and ranges therebetween; and a WBC count that is 10 to 99-fold lower, including values and ranges therebetween, than starting whole blood sample from same subject.

In accordance with the objective of the present disclosure, further provided herein is a method for preparing the GFC from conventional PRP or the PRP of the present disclosure. Particularly preferred is an embodiment wherein the GFC is prepared from the PRP of the present disclosure. Said PRP of the present disclosure, in non-limiting embodiments, is prepared by the afore-mentioned method.

Accordingly, the present disclosure provides a method for preparing a growth factor concentrate (GFC) obtained from the PRP prepared according to the methods described herein. That is, in some embodiments, the platelet-derived growth factor concentrate (GFC) of the present disclosure is prepared from a PRP, wherein the PRP has a platelet count that is about 10 to 20-fold greater than starting whole blood sample, or a RBC count that is about 60 to 90-fold lower than starting whole blood sample, and/or a WBC count that is about 10 to 99-fold lower than starting whole blood sample.

While the GFC of the present disclosure is prepared from the PRP of the present disclosure, the method for preparing which is described above, it will be understood by a person skilled in the art that similar steps can be applied to conventional PRP for obtaining GFC therefrom. To prepare the GFC, platelets present in the PRP are activated by subjecting the PRP to one or more platelet-activating treatments.

In preferred embodiments of the present disclosure, to prepare the GFC, platelets present in the PRP of the present disclosure are activated by subjecting the PRP to one or more platelet-activating treatments.

The platelet-activating treatment is selected from a group comprising treatment with platelet activation buffer and freeze-thaw cycles or a combination thereof.

In some embodiments, the platelet activation buffer comprises platelet activating agent selected from a group comprising collagen, calcium salt, hyaluronic acid, thrombin, and any combination thereof. In exemplary embodiments, the platelet-activating treatment comprises acombination of treatment with platelet activation buffer and one or more freeze-thaw cycles. In some embodiments, the PRP is treated with platelet activation buffer and said treated PRP is subsequently subjected to one or more freeze-thaw cycles. In some embodiments, the freeze-thaw cycles may precede the treatment with platelet activation buffer. In some other embodiments, the PRP may be subjected to alternating treatment with platelet activation buffer and one or more freeze-thaw cycles. In further embodiments, the PRP may be subjected to treatment with platelet activation buffer and freeze-thaw cycles simultaneously.

In some embodiments, the platelet activating agents such as collagen, a calcium salt, hyaluronic acid, thrombin, or any combination of two or more thereof are provided in a physiologically suitable buffer. In some embodiments, the platelet activating treatment comprises incubating the PRP, for about 15-45 minutes, with a buffer comprising collagen, a calcium salt, and hyaluronic acid. In some embodiments, the platelet activating treatment comprises incubating PRP, for about 15-45 minutes, with a buffer comprising collagen, hyaluronic acid, and thrombin. In some embodiments, the platelet activating treatment comprises incubating PRP, for about 15-45 minutes, with a buffer comprising a calcium salt, hyaluronic acid, and thrombin. In some embodiments, the platelet activating treatment comprises incubating PRP, for about 15-45 minutes, with a buffer comprising a calcium salt and hyaluronic acid followed by subjecting the PRP to freeze-thaw cycles. In some embodiments, the platelet activating treatment comprises incubating PRP, for about 15-45 minutes, with a buffer comprising collagen and hyaluronic acid followed by subjecting the PRP to freeze-thaw cycles. In some embodiments, the platelet activating treatment comprises incubating PRP, for about 15-45 minutes, with a buffer comprising thrombin and hyaluronic acid followed by subjecting the PRP to freeze-thaw cycles. In some embodiments, the platelet activating treatment comprises incubating PRP, for about 15-45 minutes, with a buffer comprising a calcium salt and thrombin followed by subjecting the PRP to freeze-thaw cycles. In some embodiments, about 10% to 30% by volume of a buffer containing platelet-activating agents is added to PRP. For example,about 100 microliter of the buffer containing platelet-activating agents is added to 1 ml of PRP.

In some embodiments, the PRP incubated with a buffer containing platelet-activating agents is subjected to 2-7 freeze-thaw cycles. A freeze-thaw cycle comprises freezing the PRP incubated with one or more platelet-activating agents to about 4° C., 20° C., or 80° C., and thawing the frozen PRP at a temperature of about 20° C. to 37° C. or about 25° C. to 37° C. The PRP upon treatment with a platelet-activating treatment forms a gel-like consistency. The gel upon standing separates spontaneously from liquid supernatant. The supernatant contains the GFC.

In some embodiments, the method for preparing GFC comprises: (a) incubating a whole blood sample collected in an anti-coagulant container with RBC aggregating agent(s); (b) subjecting the whole blood sample incubated with the RBC aggregating agent to a first centrifugation step to obtain a supernatant containing platelets; (c) subjecting the supernatant to a second centrifugation step to obtain a platelet pellet and platelet-poor plasma (PPP); and (d) re-suspending the platelet pellet in PPP to obtain the PRP; (e) subjecting the PRP to platelet-activating treatment; and (f) collecting supernatant containing the growth factor concentrate.

In some embodiments, the method for preparing GFC comprises (a) incubating a whole blood sample collected in an anti-coagulant container with RBC aggregating agent(s) selected from a group comprising heparin, collagen, a calcium salt, hyaluronic acid, polygeline, thrombin, gelatin, EDTA, sodium citrate, starch, and any combination thereof, wherein the incubation is carried out at a temperature of about 20° C. to25° C.; (b) subjecting the whole blood sample incubated with the RBC aggregating agent to a first centrifugation step to obtain a supernatant containing platelets, wherein the first centrifugation is carried out at about 300 rpm to1000 rpm for about 2 minutes to10 minutes; (c) subjecting the supernatant to a second centrifugation step to obtain a platelet pellet and platelet-poor plasma (PPP), wherein the second centrifugation is carried out at about 900 rpm to 4000 rpm for about 5 minutes to 15 minutes; and (d) re-suspending the platelet pellet in PPP to obtain the PRP (e) activating platelets in the PRP by subjecting the PRP to a platelet-activating treatment selected from a group comprising treatment with platelet activation buffer and free-thaw cycles or a combination thereof, wherein the platelet activation buffer comprises platelet activating agent selected from a group comprising collagen, a calcium salt, hyaluronic acid, thrombin, and any combination thereof; and (f) collecting supernatant containing the growth factor concentrate.

The above described order of steps is not binding on the method of the present disclosure and does not restrict the order in which the steps must be performed. The steps may be performed in any order that is logically feasible. The possibility of supplementing the methods of the present disclosure with steps/modifications routinely practiced in the art in relation to preparation of PRP and platelet derived growth factor compositions is envisaged by the present disclosure.

Once obtained, the platelet-derived growth factor concentrate (GFC) can be put to application instantly or may be subjected to storage for subsequent use. In a non-limiting embodiment, the GFC is stored in airtight vials. Storage without diminished quality is feasible for a period of about 3 to 6 months at a storage temperature ranging from about minus 196 degree Celsius to 4 degree Celsius.

As mentioned above, when put to application, the platelet derived growth factor concentrate of the present disclosure may be employed independently or in combination with the PBSCs or endothelial cells. Thus, in order to further the application of the above described product(s), the present disclosure provides a therapeutic composition comprising the GFC, a thermoresponsive polymer and optionally, the PBSCs and/or additional active agents.

The present disclosure thus provides a method for preparing the therapeutic composition comprising a thermoresponsive polymer; GFC obtained from either conventional PRP or PRP prepared by the present disclosure; optionally along with peripheral blood stem cells (PBSCs), and one or more additional therapeutic agents. The method comprises mixing GFC derived from conventional or PRP of the present disclosure with the thermoresponsive polymer, optionally along with the PBSCs and additional therapeutic agents, to obtain the composition.

In some embodiments, the mixing of the components to prepare the composition of the present disclosure is carried out by adding the GFC at a concentration ranging from about 10% to 50% directly to the thermoresponsive polymer under sterile environment. While this thermoresponsive polymer is prepared separately in a liquid selected from water or saline, such as PBS, prior to its mixing with the GFC, it is important to note that the concentration of the thermoresponsive polymer must also remain between about 10% to 50% in the final therapeutic composition of the present disclosure.

In some embodiments, depending on the end application of the therapeutic compositions of the present disclosure, the thermoresponsive polymer employed to prepare the composition is in the form of a powder, which is subjected to mixing with water or saline, including PBS, to form a liquid. This liquid is subsequently mixed with the GFC to obtain the composition of the present disclosure. However, in alternative embodiments, the thermoresponsive polymer may remain in the form of a powder and mixed directly with the GFC to obtain the composition of the present disclosure. In any case, the concentrations of the thermoresponsive polymer within the compositions herein remain within the range provided in the disclosure herein.

In some embodiments, a method for preparing a polymer solution as mentioned above comprises steps of: a) combining an amount of the thermoresponsive polymer or a combination of two polymers (such as NIPAM and PEG) with an amount of a suitable aqueous solvent fortified with growth factors, wherein the amount of polymer(s) is sufficient to form a solution having up to about 10% to 50% w/w of polymer(s); b) stirring the mixture at a sufficiently medium speed at about or below 10° C. for a first period of time; and c) rocking the mixture for a second period of time thereby forming a solution.

Post contacting of the thermoresponsive polymer with the GFC, the mixture is cooled in refrigerator or over ice at a temperature ranging from about 2° C. to 10° C. for about 15 minutes. The tube is periodically shaken to help mixing of the contents. Upon dissolving, the mixture is allowed to settle for elimination of air bubbles, post which the mixture, or the composition, is ready for therapeutic administration.

Since the compositions herein also contemplate inclusion of PBSCs or the endothelial progenitor cells and/or one or more additional therapeutic agents, the present disclosure also provides for methods for said inclusion(s) accordingly. In non-limiting embodiments, without being bound to said order alone, the GFC is combined with the PBSCs and the one or more additional therapeutic agents and said combination is thereafter added to the polymer, which is preferably in the form of a liquid gel.

In some embodiments, the PBSCs are incorporated into the compositions of the present disclosure comprising the thermoresponsive polymer and GFC just prior to administration of the said composition to a subject have low thickness or receptivity of the endometrial lining.

In some embodiments, the PBSCs are mixed with the GFC, followed by which said combination is mixed with the thermoresponsive polymer prior to administration of the said composition to a subject have low thickness or receptivity of the endometrial lining.

In order to facilitate preparation of said composition, the present disclosure further provides a method for the preparation of PBSCs. In some embodiments of the present disclosure, once the whole blood is collected for the preparation of the PRP or the GFC, a fraction of the blood is kept aside for the preparation of endothelial progenitor cells or PBSCs.

As mentioned previously, since the subject is administered G-CSF one to three days prior to the administration of the composition, the Bone-Marrow Derived Stem Cells (BMSCs) are mobilized leading to circulation of the PBSCs in the blood.

On the day of the administration, the said blood is withdrawn and subjected to conventional protocols for harvesting of the PBSCs. The said conventional protocols include those that provide for removal of PBSCs by buffy coat preparation.

Accordingly, in some embodiments, the PBSCs are prepared in a solution form by the following buffy coat protocol comprising steps of:
a) storing the whole blood at a temperature ranging from about 20° C. to 24° C., followed by centrifugation at 1500rpm/10 minutes
b) allowing formation of three layers as per cell density of the blood, comprising a bottom layer consisting of RBCs, a middle layer consisting of platelets and WBCs, and a top layer comprising platelet poor plasma (PPP); and
c) removing the supernatant plasma from the top layer (buffy coat) and transferring the buffy-coat layer to another sterile tube, followed by centrifugation at 2000 rpm/12 minutes or filtration to separate WBCs and obtain the solution comprising PBSCs.

In a non-limiting embodiment, the centrifugation at high speed comprises centrifugation at about 2000 rpm to 5000rpm, for about 10 mins to 20 minutes, preferably at about 2000 rpm for about 15 minutes. In another non-limiting embodiment, the centrifugation at low speed comprises centrifugation at about 100 rpm to 1500 rpm, for about 5 to 10 mins, preferably at about 200rpm for about 10 minutes.

Once the solution comprising the PBSCs is prepared, it is mixed with the GFC and the thermoresponsive polymer, said mixing of components being restricted by no particular order, such that the growth factor concentrate, the PBSCs, and thermoresponsive polymer are present at a ratio of about 30:30:40 to 40:40:20.

In some embodiments of the present disclosure, the composition further comprises additional therapeutic agent(s) selected from a group comprising hormone, growth factor, protein, cell, cell secretome, and drug, or any combination thereof.

In exemplary embodiments, the additional therapeutic agent(s) is selected from a group comprising Vitamin E, human chorionic gonadotropin(HCG), leukemia inhibitory factor (LIF),

Vascular endothelial growth factor (VEGF), Metalloproteinase-9 (MMP-9), Aspirin, Heparin, Sildenafil citrate, estrogen, progesterone, Stem cells, Cells/Stem cell secretome.

In some embodiments, in addition to growth factors from autologous blood, the therapeutic composition is further fortified with exogenously added growth factors to provide a concentration of growth factors that is about 4 to 10 times higher than the baseline concentration of corresponding growth factors in starting whole blood. In some embodiments,the composition is fortified with growth factor(s) selected from a group comprising TGF, EGF, IGF-1, bFGF, PDGF, LIF, VEGF, SCF, IL-1b, Fibronectin, IL-1, CSF, HIF-alpha, Activin A, IL-8, TNF-a, NF-kB and any combination thereof.

In non-limiting embodiments, the additional therapeutic agent(s) and/or fortifying growth factor(s) are added to the combination of growth factor concentrate, the thermoresponsive polymer and optionally, the PBSCs, to obtain the composition of the present disclosure.

It is to be noted that while preparing the compositions of the present disclosure, the thermoresponsive polymer is the last component added to the composition just prior to administration of the composition. That is all components including GFC and optional components like PBSCs and additional therapeutic agents are mixed and the thermoresponsive polymer is added in the end just prior to administration.

Accordingly, in some embodiments, in the therapeutic compositions, concentration of the GFs ranges from about VEGF ranges from about 500-1300 pg/mL, concentration of the EGF ranges from about 100-2000 pg/mL, concentration of the bFGF ranges from about 25-500 pg/mL, concentration of the IGF-1 ranges from about 500-1000 ng/mL, concentration of the PDGF-BB ranges from about 20-500 ng/mL, and concentration of the TGF-b1 ranges from about 100-2000 ng/mL.

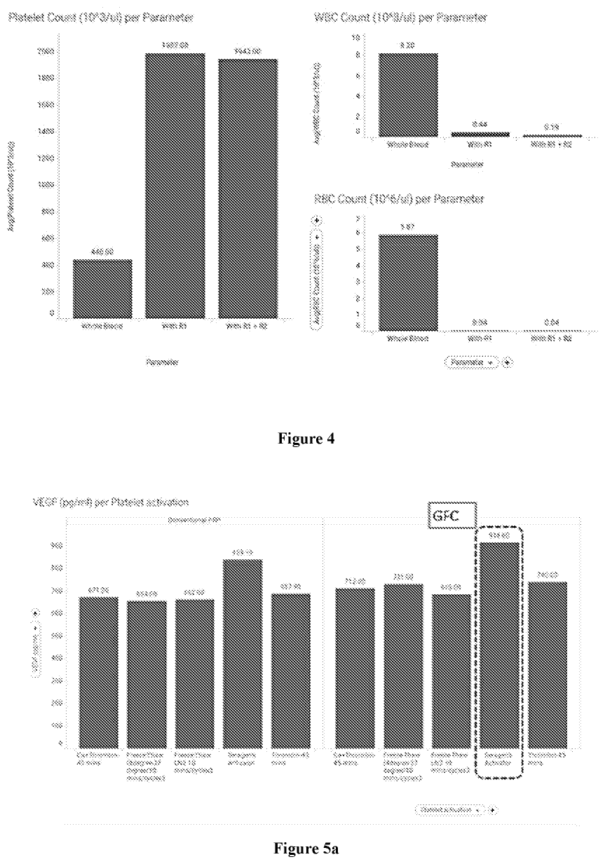

The present disclosure further provides a method for treating an endometrial defect causing infertility in a subject in need thereof comprising, administering to the subject the GFC or any of the therapeutic compositions described above. As an exemplary representation, the schematic scheme for preparing the composition of the present disclosure and the subsequent administration is provided in FIG. 3.

In some embodiments, the administration is repeated one or more times.

In non-limiting embodiments, said administration occurs over a 15-day cycle keeping track of the menstrual cycle, typically with 3 repeat doses. The first dose is administered on day 4, 5 or 6 of the cycle, the second dose is administered 5 days from the first dose and the 3^{rd} dose is administered 2 days before embryo injection.

In non-limiting embodiments, the composition or the GFC is administered through intrauterine injections or hysteroscopic sub-endometrial injection. All other modes of administration that would facilitate delivery of the composition or the GFC to the uterus or the endometrium are envisaged in the scope of this disclosure.

Administration of the composition has been shown to improve endometrial lining thickness in patients with non-responsive thin endometrium less than 7 mm

In some embodiments, the present disclosure relates to the GFC or the therapeutic composition for use in preparing a medicament to improve endometrial thickness and receptivity in IVF procedure.

In some embodiments, the present disclosure relates to use of the GFC or the therapeutic composition to increase endometrial thickness and receptivity to facilitate embryo implantation in ART.

In some embodiments, the present disclosure relates to use of the GFC or the therapeutic composition to treat infertility due to reduced endometrial thickness and receptivity.

Now, in order to facilitate preparation of the GFC of the present disclosure, and subsequently the compositions comprising the GFC, the present disclosure also provides a kit.

Thus, the present disclosure provides a kit for preparing the therapeutic compositions of the present disclosure, wherein the kit as comprises:
a. RBC activating agent selected from a group comprising: heparin, collagen, a calcium salt, hyaluronic acid, polygeline, thrombin, gelatin, EDTA, sodium citrate, starch, and any combination thereof;
b. thermoresponsive polymer(s); and
c. an instruction manual.

In some embodiments, the kit further comprises G-CSF. Said G-CSF has a role to play in stem cell mobilization before withdrawal of blood for the preparation of the GFC.

In some embodiments, the kit of the present disclosure further comprises a platelet activating agent selected from a group comprising: collagen, a calcium salt, hyaluronic acid, and thrombin, or a combination thereof. The kit also comprises a blood collection container comprising an anti-coagulant.

In some embodiments, the kit of the present disclosure further comprises additional therapeutic agent(s) selected from a group comprising hormone, growth factor, protein, cell, cell secretome, and drug, or any combination thereof. In preferred embodiments, the additional therapeutic agent(s) is selected from a group comprising Vitamin E, human chorionic gonadotropin (HCCG), leukemia inhibitory factor (LIF), Vascular endothelial growth factor (VEGF), Metalloproteinase-9 (MMP-9), Aspirin, Heparin, Sildenafil citrate, estrogen, progesterone, Stem cells, Cells/Stem cell secretome.

In some embodiments, the kit further comprises growth factor(s) to fortify the composition. In non-limiting embodiments, the growth factor(s) incorporated in the kit is selected from a group comprising TGF, EGF, 1-IGF-1, bFGF, PDGF, LIF and any combination thereof.

As is clear, the kit of the present disclosure is used for preparing the therapeutic compositions herein. In other words, the kit of the present disclosure allows for:
a. processing of whole blood for preparation of PRP of the present disclosure;
b. processing of whole blood for preparation of GFC from the PRP of the present disclosure;
c. processing of conventional PRP for preparation of GFC of the present disclosure; and/or
d. preparation of the therapeutic compositions of the present disclosure comprising GFC and thermosensitive polymer.

Since the kit comprises the RBC activating agent, in some embodiments, the kit also facilitates preparation of PBSCs for inclusion in the compositions of the present disclosure. Accordingly, the kit of the present disclosure also allows for:
a. preparing the therapeutic compositions of the present disclosure comprising PRP and thermosensitive polymer, and PBSCs; and
b. preparing the therapeutic compositions of the present disclosure comprising GFC and thermosensitive polymer, and PBSCs.

Further, since the kit comprises one or more additional therapeutic agent(s), in some embodiments, the kit also facilitates preparation of the compositions of the present disclosure having said additional therapeutic agent. In other words, the kit facilitates preparation of the therapeutic compositions of the present disclosure comprising GFC and thermosensitive polymer, and optionally PBSCs and/or additional therapeutic agents.

In some embodiments, the kit comprises an instruction manual having steps for: processing of the whole blood for processing of whole blood for preparation of PRP of the present disclosure; processing of whole blood for preparation of GFC from the PRP of the present disclosure; processing of conventional PRP for preparation of GFC of the present disclosure; preparing of the therapeutic compositions of the present disclosure comprising PRP and thermosensitive polymer; and/or preparing of the therapeutic compositions of the present disclosure comprising GFC and thermosensitive polymer. The instructional manual may additionally comprise steps for processing of PBSCs and/or inclusion on additional therapeutic agent during preparation of any of the said compositions.

While the instant disclosure is susceptible to various modifications and alternative forms, specific aspects thereof have been shown by way of examples and drawings and are described in detail below. However, it should be understood that it is not intended to limit the invention to the particular forms disclosed, but on the contrary, the intention is to cover all modifications, equivalents, and alternatives falling within the spirit and the scope of the invention as defined by the appended claims.

### EXAMPLES

### Example 1

### Preparation of Platelet-Rich Plasma (PRP)

30 ml of venous blood was drawn from a patient and 10 ml each was aliquoted into acid citrate dextrose (ACD-A) solution gel tube/K2 EDTA tube. The samples were incubated for 45 minutes with a buffer comprising polygeline, gelatin, and starch as RBC aggregating agents. After incubation, samples were centrifuged at 600 rpm for 2 minutes. Supernatant containing platelets was collected and again centrifuged at 3000 rpm for 12 minutes. After this centrifugation, platelets sedimented as a pellet and the supernatant contained platelet-poor plasma (PPP). The platelet pellet was re-suspended in 3 ml of PPP to obtain PRP.

The number of platelets, RBCs, and WBCs in the PRP were counted. The Table below shows the cell count obtained by the above-described method (PRP of the present disclosure) and comparative cell count obtained by conventional PRP methods. The cell count values for conventional PRP methods are based on the values disclosed in Principles and Methods of Preparation of Platelet-Rich Plasma: A Review and Author's Perspective J Cutan Aesthet Surg. 2014 October-December; 7(4): 189-197.doi: 10.4103/0974-2077.

TABLE 2

| | | Platelets | | Total WBC | RBC |
|---|---|---|---|---|---|
| | | Count | Fold increase | Count | Count |
| | Parameters | 10\hat{ }3/ul | over whole blood | 10\hat{ }3/ul) | (10\hat{ }6/ul) |
| 1 | Whole Blood (Minimum | 150 | — | 4.5 | 4.7 |
| | Normal Value) | | | | |
| 2 | Conventional PRP Protocol | 1096 | 7.4 | 12.6 | 8.9 |
| | (Single Spin/Buffy | | | | |
| | Coat Method) | | | | |
| 3 | Conventional PRP Protocol | 1577 | 10.5 | 11.3 | 1.1 |
| | (DoubleSpin/PRP method) | | | | |
| 4 | PRP Method of the | 2023 | 13.4 (1.8 | 0.19 (23.6 | 0.06 (78.33 |
| | present disclosure | | fold over | fold | fold |
| | | | single spin/ | reductionover | reduction over |
| | | | 1.3 fold | whole blood/ | whole blood/ |
| | | | over double | 66.3 fold | 148.3 fold |
| | | | spin) | reductionover | reduction over |
| | | | | single spin/ | single spin/ |
| | | | | 59.47 fold | 18.33 fold |
| | | | | reduction | reduction over |
| | | | | overdouble | double |
| | | | | spin) | spin) |

### Example 2

### Preparation of Platelet-Derived Growth Factor Concentrate (GFC)

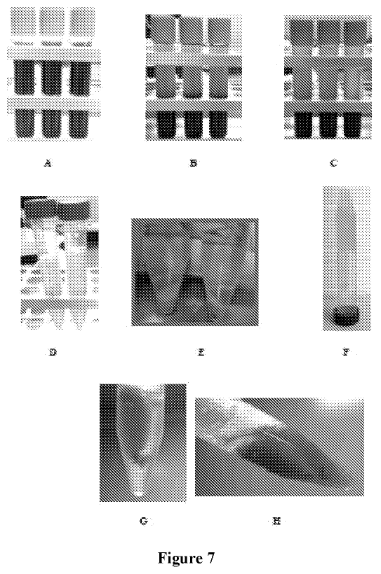

PRP was prepared as described in Example 1. 300 μl of a platelet activation buffer comprising calcium chloride (10%) and thrombin (10%) was mixed with the PRP (at 10% concentration of the mixture of calcium chloride and thrombin) and the mixture was incubated for 45 minutes. After incubation, the mixture was subjected to three freeze-thaw cycles with freezing at 4° C. and thawing at 37° C. The supernatant containing the GFC was collected and aliquoted into cryovials, which can be used for administration right away orcan be preserved for future use. FIG. 7 panels A-H represent the images of various stages ofwhole blood processing for preparing the PRP and the GFC of the present disclosure.

ELISA assays were performed to determine levels of growth factors present in the freshly-prepared GFC and the levels upon storage at 20° C. or −10° C. The Table below shows the levels in the freshly-prepared GFC and the levels upon storage at 20° C. for a duration of 3, 6, 9, and 12 hours.

TABLE 3
Growth factor profile of freshly-prepared GFC and GFC upon storage at 20° C.

| | pg/ml | pg/ml | pg/ml | ng/ml | ng/ml | ng/ml |
|---|---|---|---|---|---|---|
| Duration | VEGF | EGF | bFGF | IGF-1 | PDGF-BB | TGF-b1 |
| Fresh | 914 ± 400 | 183 ± 50 | 50.2 ± 24.0 | 102.7 ± 26.5 | 53.2 ± 32.3 | 294 ± 45.2 |
| 1 h | 901 ± 390 | 190.2 ± 34.2 | 54 ± 22.7 | 108.5 ± 28.4 | 60.2 ± 22.4 | 310.2 ± 34.2 |
| 3 h | 850.2 ± 381.2 | 178 ± 43.2 | 47 ± 21.4 | 98.7 ± 26.5 | 57 ± 21.4 | 280 ± 48.2 |
| 6 h | 839.1 ± 390.6 | 160 ± 46.2 | 45 ± 23.5 | 93.7 ± 25.5 | 43 ± 27.5 | 290 ± 46.2 |
| 9 h | 222.4 ± 45.3 | 65 ± 22.4 | 19 ± 10.5 | 22.3 ± 18.2 | 21 ± 11.5 | 135 ± 23.4 |
| 12 h | 112 ± 45.3 | 46 ± 20.4 | 18 ± 23.5 | 24.4 ± 17.5 | 14 ± 13.5 | 60.2 ± 22.4 |

The Table below shows the levels in the freshly-prepared GFC and the levels upon storage at −10° C. for a duration of 1 week, 4 weeks, 8 weeks, 12 weeks and 24 weeks.

TABLE 4
Growth factor profile of Freshly-prepared GFC
and GFC upon storage at −10° C.

| | VEGF | EGF | bFGF | IGF-1 | PDGF-BB | TGF-b1 |
|---|---|---|---|---|---|---|
| Duration | pg/ml | pg/ml | pg/ml | ng/ml | ng/ml | ng/ml |
| Fresh | 914 | 183 | 50.2 | 102.7 | 53.2 | 294 |
| 1 w | 890 | 190 | 58 | 110 | 62 | 260 |
| 4 w | 850.2 | 210 | 51 | 97 | 56 | 280 |
| 8 w | 839.1 | 170 | 47 | 93.7 | 43 | 290 |
| 12 w | 890 | 200 | 50 | 82 | 49 | 240 |
| 24 w | 860 | 160 | 46 | 96 | 51 | 270 |

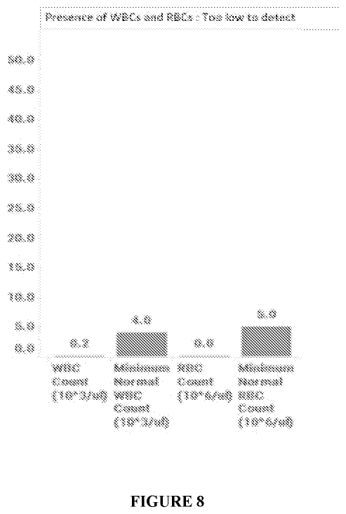

FIG. 8 shows a comparison of the RBC and WBC count between the GFC of the present disclosure and the starting whole blood wherein the RBC and WBC counts of the GFC are near negligible as compared to whole blood.

### Example 3

### Preparation of Peripheral Blood Stem Cells (PBSCs)

10 ml of venous blood was drawn from a patient into an acid citrate dextrose (ACD-A) solution gel tube/K2 EDTA tube. The sample was incubated for 45 minutes with a buffer comprising polygeline, gelatin, and starch as RBC aggregating agents. After incubation, samples were centrifuged at 1500 rpm for 10 minutes. Upon centrifugation, RBCs, WBCs, and platelets were separated as follows: the bottom layer contained RBCs, the middle layer contained platelets and WBCs (buffy coat layer) and the top layer was platelet-poor plasma. The top layer (PPP) was removed and the middle buffy coat layer was transferred to another sterile tube. The tube was centrifuge at 2000 rpm for 12 minutes to separate WBCs. Alternatively, leucocyte filtration filter can be used to separate WBCs. The Table below shows the WBC, RBC, and platelet count of the PBSC solution obtained using this method. The numbers in parenthesis in the last column indicate fold increase over whole blood.

TABLE 5
Cell count of PBSCs

| | Whole blood | Buffy | |
|---|---|---|---|
| Parameters | (Range) | coat/PBSCs | |
| WBC (×10\hat{ }3/ul) | 1.44-30.75 | 5 | (5x) |
| RBC (×10\hat{ }6/ul) | 1.66-5.96 | 1.0 | |
| PLT (×10\hat{ }3/ul) | 150-450 | 690 | (>4x) |

### Example 4

### Preparation of Composition Comprising GFC and Thermoresponsive Polymer

For preparing a composition comprising GFC and thermoresponsive polymer RNIPAM based polymer-poly(Nisopropylacrylamide-co-n-butyl methacrylate) poly(NIPAAm-co-BMA)1, the first step was to obtain the GFC. As described in the present disclosure, the GFC can either be obtained from conventionally known PRP, or by specific protocol as recited in example 1 above.

In the present example, the objective was to prepare 0.8 ml of the composition for administration into uterus of a subject in need thereof. Accordingly, about 0.4 ml of the GFC prepared by the exemplified protocol was taken for mixing with 0.4 ml or 50% (as a final concentration) of the thermoresponsive polymer

Separately, the thermoresponsive polymer, which was in the form of a powder, was subjected to mixing with water or saline to form a solution having a concentration of about 50%. For this, the following steps were performed:
d. the thermoresponsive polymer was dissolved in 50 ml amount of water to obtain a solution having up to about 50% w/w of polymer(s);
e. the solution was stirred at medium speed of about 30 rpm to 100 rpm at about 10° C. at for a first period of time of about 15 minutes; and
f. the mixture was rocked for a second period of time of about 15 minutes thereby forming a solution.

In an alternate experiment, the thermoresponsive polymer, was directly taken in the form of a powder for mixing with the GFC, without dissolution in water or saline.

Accordingly, two batches of mixtures were prepared. One comprising about 0.4 ml of the GFC and 0.4 ml of the solution of the polymer; and the second comprising about 0.4 ml of the GFC and 0.4 ml of the polymer powder (50%). For preparation of these mixtures, the following steps were performed:
a. the thermoresponsive polymer was contacted with the GFC in a sterile tube, and the mixture was cooled in refrigerator at a temperature of about 8° C. for about 10 minutes;
b. the tube was periodically shaken to help mixing of the contents and maintained at the same temperature;
c. once dissolved, the mixture was allowed to settle for elimination of air bubbles.

This mixture comprised of 0.4 ml of GFC and 0.4 ml or 50% of the thermoresponsive polymer.

This experiment was subsequently repeated by replacing the NIPAM based polymer with Poloxamer 407 to obtain a composition comprising GFC and Poloxamer 407.

These final compositions were prepared for administration to a subject having thin endometrial lining

### Example 5

### Preparation of Composition Comprising GFC and Thermoresponsive Polymer Along with PBSCs

For preparing a composition comprising GFC and thermoresponsive polymer [(NIPAM based polymer poly(Nisopropylacrylamide-co-n-butyl methacrylate) poly(NIPAAm-co-BMA)], along with PBSCs, the first step was to obtain the GFC. As described in the present disclosure, the GFC can either be obtained from conventionally known PRP, or by specific protocol as recited in example 2 above.

In the present example, the objective was to prepare 1 ml of the composition for administration into uterus of a subject. Accordingly, about 0.30 ml of the GFC prepared by the exemplified protocol was taken for mixing with 0.20 ml or 20% (as a final concentration) of the thermoresponsive polymer.

Separately, four batches of the thermoresponsive polymer were prepared two in solution form (similar to example 4 above) and two directly in the powder form.

Separately, four fractions of 0.50 ml (50% of the final composition) of the PBSCs were prepared from the whole blood of the subject, as per the buffy coat protocol described in example 3.

For preparing the final compositions, four batches of initial mixtures were prepared, that comprised of PRP or GFC and PBSCs for final mixing with the polymer as follows:
PRP and PBSC for mixing with polymer in powder form;
PRP and PBSC for mixing with polymer in solution form;
GFC and PBSC for mixing with polymer in powder form; and
GFC and PBSC for mixing with polymer in solution form.

Each of these batches comprised of about 0.30 ml of the PRP or GFC respectively and about 0.50 ml or 50% of the PBSCs. For preparation of these mixtures, simple mixing steps were carried out.

To these 4 batches, the 4 fractions of 0.20 ml (20%) of the polymer was added, to prepare the final composition for administration to a subject having low thickness of endometrial lining. For preparation of these final mixtures, mixing steps similar to those in example 4 were followed. The following table 6 provides for the particulars of the composition prepared herein:

TABLE 6

| Particulars | Uterus |
|---|---|
| Cells/Stem cells (V %) | 50 |
| ABCD/PRP (V %) | 30 |
| Polymer (V %) | 20 |
| Final volume (ml) | 1 |

This experiment was subsequently repeated by replacing the NIPAM based polymer with Poloxamer 407 to obtain a composition comprising PRP or GFC, Poloxamer 407 and PBSCs.

### Example 6

### Preparation of Composition Comprising GFC and Thermoresponsive Polymer with Additional Therapeutic Agent

For preparing a composition comprising PRP or GFC and thermoresponsive polymer [(NIPAM based polymer poly(Nisopropylacrylamide-co-n-butyl methacrylate) poly(NIPAAm-co-BMA) or Poloxamer 407], with or without PBSCs, and with additional therapeutic agent, the overall protocol remained the same as those described in the previous examples. The additional therapeutic agent was added to a mixture of GFC and PBSCs and said combination was added to the polymer in its liquid gel state.

### Example 7

### Effect of Thermoresponsive Polymer on Release Profile of the Composition Comprising Recombinant Growth Factor

This example was designed for assessing the importance of the thermoresponsive polymer in the compositions of the present disclosure. This was carried out by comparing the growth factor release profile from a composition comprising the polymer, and a composition devoid of it. For further analysis on the effect of the polymer, regardless of the underlying active component, a test composition of recombinantly prepared VEGF with the polymer was also prepared.

In this example, a test composition of recombinant VEGF with the polymer, was prepared by a simple 1:1 mixing of the recombinant VEGF with the polymer.

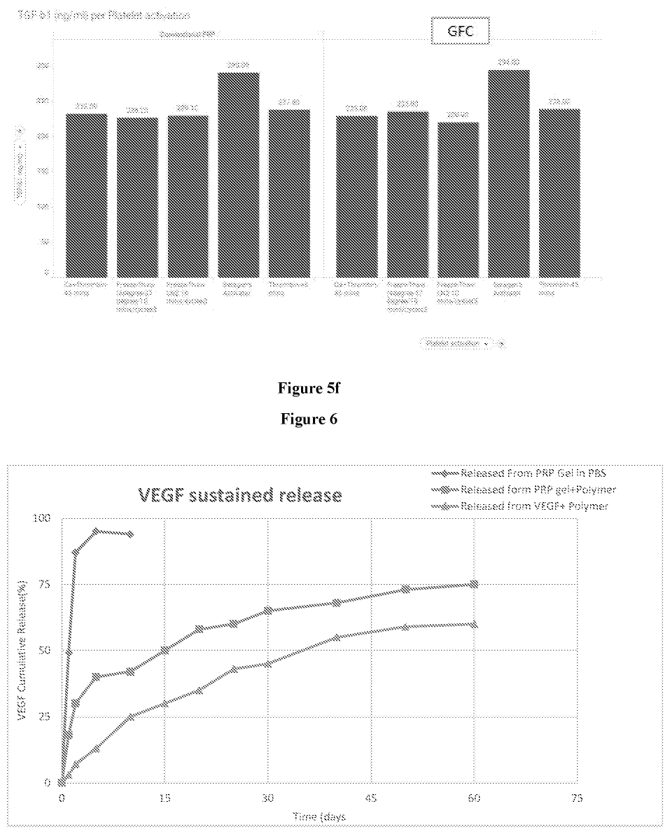

The in vitro growth factor release kinetics was performed in PBS (pH 7.4) at 37° C. for 60 days as reported in FIG. 6. As can be seen, VEGF released from PRP mixed with polymer within the first 2 days (burst effect) was 30±3%, followed by a phase of sustained release with almost 75% of VEGF being released within 60 days (orange/middle graph). Although, the VEGF release was lower for composition of recombinant VEGF mixed with polymer, it still showed good profiling over the full 60 day period (gray/third graph from top). However, in contrast, no release of growth factors was observed for the preparation of PRP in PBS beyond the first 10 days (blue/first graph from top). Accordingly, it is evident that the composition devoid of the polymer lost any ability for sustained effect because of the dilution. However, very clearly, the polymer supports the sustained delivery of growth factors in both the compositions that had it. The growth factor release from the polymer validates the slow releaseof these proteins for long term availability and therapeutic efficacy.

### Example 8

### Analysis of the Effect of RBC Aggregators on the GFC Profile

Example 1 was repeated with the following variations
A) Employment of a single RBC aggregator—Gelatin
B) Employment of a combination of 2 RBC aggregators—Gelatin and Starch
C) Employment of no RBC aggregator
D)-F) No RBC aggregators

Experiments A-F were designed to have gradually increasing centrifugation speed and time. G was a control experiment.

Specifics of the above experiments are depicted in Table 7 below.

TABLE 7
Blood Processing for GFC - Protocol Standardization

| Step | Parameter | A | B | C | D | E | F | G |
|---|---|---|---|---|---|---|---|---|
| 1 | RBC | With | With | Whole | | | | |
| | aggregators | RBC1 | RBC1 + 2 | Blood | | | | |
| 2 | Incubation | 15 | 30 | 45 | No | | | |
| | time-minutes | | | | | | | |
| 3 | Centrifugation- | 500 | 600 | 700 | 800 | 900 | 1000 | No |
| | rpm | | | | | | | |
| 4 | Centrifugation- | 2 | 4 | 6 | 8 | 10 | | |
| | time-minutes | | | | | | | |
| 5 | Platelet | Ca Salt- | Thrombin- | Ca + | Freeze-Thaw | Freeze- | | |
| | activation | 45 mins | 45 mins | Thrombin- | (4degree- | Thaw LN2 | | |
| | | | | 45 mins | 37degree/ | 10 mins/ | | |
| | | | | | 10 mins/ | cyclex3 | | |
| | | | | | cyclex3 | | | |

| | | |
|---|---|---|
| 4 | GFC assay- | 9*5 Assays |
| | ELISA | |

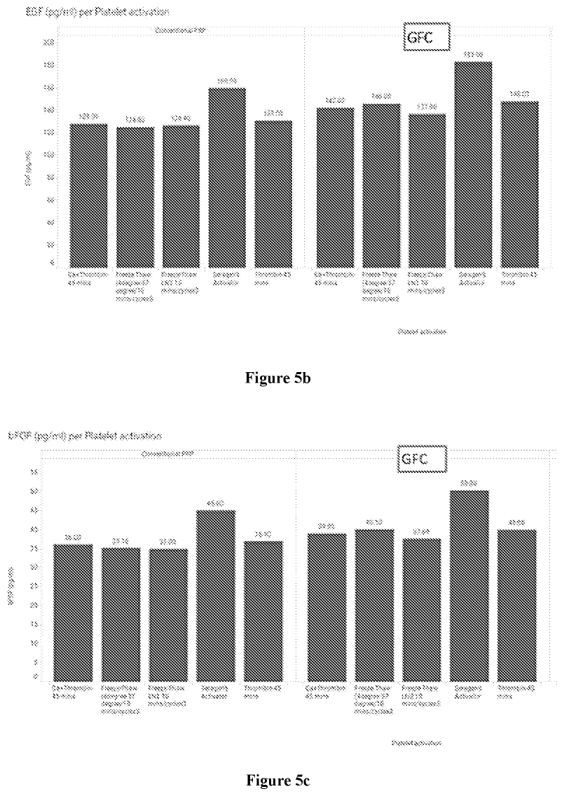

Experiments A and B which employed RBC aggregators were found to yield improved results with respect to settling and separation of RBCs and WBCs through their respective protocols. For reasons of brevity, results from variations of the experiment closest to the protocol of the present disclosure are depicted as graphs in FIG. 4. As can be observed from said figure, the incorporation of RBC aggregators in the PRP/GFC preparation protocol has a significant impact in terms of the improvement in platelet count and reduction in RBC and WBC count. The combination of 2 RBC aggregators was found to further improve the reduction in WBC count in the PRP. Further, increasing centrifugation speed and time was not found to compensate for the absence of the RBC aggregators.

### Example 9

### Analysis of the Effect of Different Platelet Activation Protocols

The effect of the choice of platelet activation protocol on the concentration of growth factors in the final GFC was analyzed by performing variations of the experiment in Example 2. Keeping other specifics of the experiment constant, said variations employed treatment of PRP with single platelet activating agent, treatment of PRP with a combination of 2 activating agents, exposure of PRP to freeze-thaw cycles at different temperatures and a combination of treatment of PRP with activation agent and exposure to freeze-thaw cycles.

Results yielded by said experiments are provided in the table below.

TABLE 8

| | | VEGF | EGF | bFGF | IGF-1 | PDGF-BB | TGF-b1 |
|---|---|---|---|---|---|---|---|
| Platelet activation | | (pg/ml) | (pg/ml) | (pg/ml) | (ng/ml) | (ng/ml) | (ng/ml) |
| PRP of the | Activation | 740 ± 380 | 148 ± 30 | 40 ± 22 | 83.1 ± 23 | 43 ± 28.9 | 238 ± 35.6 |
| present | buffer - | | | | | | |
| disclosure | Thrombin-45 | | | | | | |
| | mins | | | | | | |
| | Activation | 712 ± 395 | 142 ± 42 | 39 ± 19 | 80.1 ± 19.2 | 41 ± 21.3 | 229 ± 31.2 |
| | buffer - Ca | | | | | | |
| | salt + Thrombin- | | | | | | |
| | 45 mins | | | | | | |
| | Freeze (4° C.) - | 731 ± 372 | 146 ± 51 | 40.1 ± 15 | 82.1 ± 14.3 | 42.5 ± 18.7 | 235 ± 29 |
| | Thaw (37° C.)/10 | | | | | | |
| | mins/cycle x3 | | | | | | |
| | Freeze | 685 ± 437 | 137 ± 40 | 37.6 ± 10 | 77 ± 21.1 | 39.9 ± 19.5 | 220 ± 25 |
| | (−196° C.)- | | | | | | |
| | Thaw(37° C.)/ | | | | | | |
| | LN2 10 | | | | | | |
| | mins/cycle x3 | | | | | | |
| | Activation | 914 ± 400 | 183 ± 50 | 50.2 ± 24.0 | 102.7 ± 26.5 | 53.2 ± 32.3 | 294 ± 45.2 |
| | buffer (Ca | | | | | | |
| | salt) + Freeze- | | | | | | |
| | Thaw cycles | | | | | | |
| Conventional | Activation | 687.9 ± 370 | 131 ± 41.3 | 36.9 ± 19.4 | 76.8 ± 24.4 | 35 ± 23.4 | 237.8 ± 41.2 |
| PRP | buffer - | | | | | | |
| | Thrombin-45 | | | | | | |
| | mins | | | | | | |
| | Activation | 671.2 ± 362 | 128 ± 43.2 | 36 ± 18.9 | 74.9 ± 19.2 | 34.4 ± 18.3 | 232 ± 38.7 |
| | buffer - Ca | | | | | | |
| | salt + Thrombin- | | | | | | |
| | 45 mins | | | | | | |
| | Freeze (4° C.) - | 654 ± 358 | 124.8 ± 35.2 | 35.1 ± 21.1 | 73 ± 14.4 | 33.5 ± 19.6 | 226.2 ± 39.2 |
| | Thaw (37° C.)/10 | | | | | | |
| | mins/cycle x3 | | | | | | |
| | Freeze | 662 ± 379 | 126.4 ± 39.1 | 35.55 ± 17.3 | 74 ± 19.5 | 33.9 ± 16.2 | 229.1 ± 42 |
| | (−196° C.)- | | | | | | |
| | Thaw(37° C.)/ | | | | | | |
| | LN2 10 | | | | | | |
| | mins/cycle x3 | | | | | | |
| | Activation | 839.1 ± 390.6 | 160 ± 46.2 | 45 ± 23.5 | 93.7 ± 25.5 | 43 ± 27.5 | 290 ± 46.2 |
| | buffer (Ca | | | | | | |
| | salt) + Freeze- | | | | | | |
| | Thaw cycles | | | | | | |

Observations from the above experiments show that a platelet activation protocol employing a combination of treatment of PRP with activation agent and exposure of PRP to freeze-thaw cycles yields GFC with significantly higher growth factor concentration said effect being observed for both the PRP of the present disclosure as well as conventional PRP.

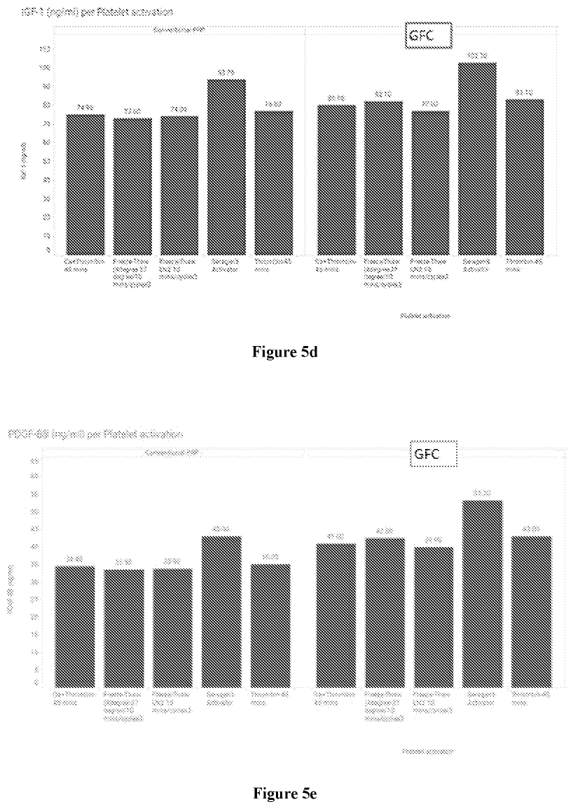

The PRP of the present disclosure, however, provides a notably higher concentration of individual growth factors in the GFC derived therefrom when compared to conventional PRP that is subjected to platelet activation by the same protocol. Thus, a synergy between the PRP preparation protocol and PRP activation protocol in yielding GFC with high growth factor concentration is derivable from the above data. The above results are depicted in FIG. 5.

### Example 10

### Analysis of the Effect of the Composition on Thickness of Endometrial Wall

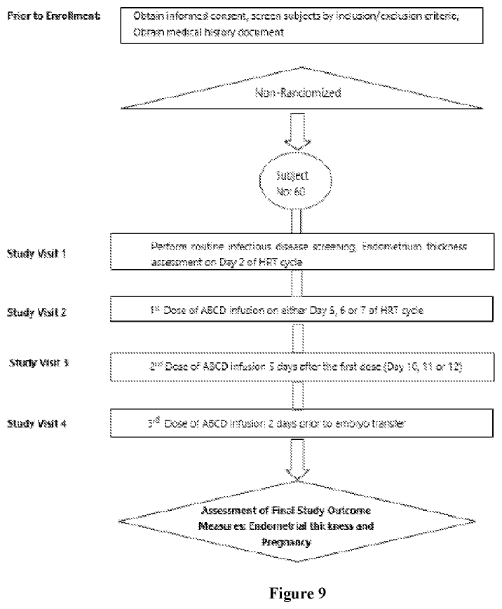

A non-randomized study for evaluating the safety and efficacy of GFC isolated from peripheral blood as done in Example 2 was conducted. It was evaluated if intrauterine administration of GFC improves endometrial lining thickness in patients with non-responsive thin endometrium less than 7mm The design of the study is depicted in FIG. 9.

Subjects were chosen because of thinning of the endometrium leading to several failed implantations. The GFC was transplanted using intrauterine method using IUI catheter or tom cat catheter.

The clinical end points were collected in the hospital every 72 hours post infusion for measuring thickness of endometrium followed by ABCD administration.

The data is represented in the table below.

TABLE 9

| | | |
|---|---|---|
| Endometrium | No of patients treated | 60 |

| | | |
|---|---|---|
| Pre PRP-ET Avg | 5 | mm |
| Improvement in ET (avg) | 7.62 | mm |
| Chemical pregnancy rate | 9 | (15%) |
| Clinical pregnancy rate | 41 | (68.33%) |
| Live Birth | 30 | (73%) |
| ectopic gestation | 3 | (0.05%) |
| Blighted Ova | 3 | (0.05%) |
| Missed abortion | 4 | (0.06%) |

In the above study, sixty patients had poor endometrial quality and the endometrium was non-responsive to conventional hormonal therapy, resulting in cycle cancellation, low possibility of pregnancy and heavily emotional distress. After application of ABCD, the endometrial thickness was satisfactory in all the patients and subsequent embryo transfer (ET) was performed. Out of sixty patients, the conditional improvement was seen in all the patients and found to be more than 7 mm which is reported to be the ideal thickness for embryo transfer.

Out of 60 patients, 41 clinical pregnancy were reported with a success rate of 73%. Out of 41 pregnancies, there were 7 miscarriages post sac development were reported and is attributed to poor quality embryo and male factor (as informed by the treating IVF expert). The overall successful clinical pregnancy is 73% as opposed to the maximum of 30%-40% success rate observed in usual IVF treatments. Interestingly, 7 pregnancies occurred at endometrial thickness less than 7 indicating the receptivity improvement.

The above data therefore shows the effectiveness of GFC in improving the endometrial regeneration and inducing the inherent regenerating capacity of endometrium.

### Example 11

### Animal Studies Effect of Incorporation of Polymer in the Composition

30 female Sprague-Dawley rats were randomly assigned into three groups: control group, ethanol group, and treated group (administration of 0.25 mL of PRP/GFC/GFC+polymer into both uterine cavities 72 hours after ethanol injection). After 15 days of endometrial damage, all the animals were sacrificed during the estrous cycle, and samples were taken from the mid-uterine horn. Functional and structural recovery of the endometrium was analyzed by hematoxylin-eosin (H&E) and Masson trichrome (MT) staining, real-time polymerase chain reaction (PCR) assay, and immuno-histochemical (IHC) analyses.

3 sets of experiments were performed on 3 separate groups of 30 rats each. The design of the experiment was as follows 1) administration of PRP of the present disclosure; 2) administration of GFC and 3) administration of GFC with polymer.

The effect of said administration on the endometrial area in said animals was observed. In order to identify clear points of comparison, within each experiment, the animals were divided into 3 sub-groups control/ethanol group/treated group.

The control group received no treatment. The ethanol group were administered with ethanol which is known to cause a reduction in thickness of the endometrial lining The third group was administered with the PRP prepared as per Example 1, GFC prepared as per Example 2 and the composition of GFC with thermoresponsive polymer prepared as per Example 4.

The results are depicted in the below table.

TABLE 10

| Condition | Parameters | PRP | GFC | GFC with Seragel |
|---|---|---|---|---|
| Endometrium | No of animals | 30 | 30 | 30 |
| | | Control/Ethanol | Control/Ethanol | Control/Ethanol |
| | | group/PRP | group/ABCD | group/ABCD + |
| | | treated ET gp | treated ET gp | Polymertreated ET gp |
| | Endometrial area | 320/205/254 | 320/205/266 | 320/205/287 |
| | (±15.77, ±15.84, ±12.33 | | | |
| | μm2, respectively) | | | |
| | Endometrial gland | +++/−/++ | +++/−/++ | +++/−/+++ |
| | Prominent nuclei | +++/−/++ | +++/−/++ | +++/−/+++ |
| | Proliferated endometrial | +++/−/++ | +++/−/++ | +++/−/+++ |
| | glands | | | |
| | Endometrial stromal cells | +++/−/++ | +++/−/++ | +++/−/+++ |
| | Collagen deposition | +++/−/++ | +++/−/++ | +++/−/+++ |
| | (% of blue stained area) | | | |
| | Stem cell marker | +++/−/++ | +++/−/++ | +++/−/+++ |
| | Pro-inflammatory marker | +++/−/++ | +++/−/++ | +++/−/+++ |
| | Anti-inflammatory marker | +++/−/++ | +++/−/++ | +++/−/+++ |

As can be observed from the table above, the composition comprising GFC and polymer led to a notable increase in endometrial area, keeping all other parameters in line with the control. A comparison between results for the PRP vs GFC group shows that the absence of cells in GFC as compared to PRP also has an impact on enhancement of the endometrial area. The best results are brought about by a combination of GFC and polymer, which establishes the synergy between said components of the composition of the present disclosure.

### Example 12

### Preparation of Kit of the Present Disclosure

A kit was prepared in accordance with the requirements of the present disclosure. The kit so prepared comprises of the following components:
a. G-CSF;
b. a RBC activating agent selected from a group comprising: heparin, collagen, a calcium salt, hyaluronic acid, polygeline, thrombin, gelatin, EDTA, sodium citrate, starch, and a combination thereof;
c. a thermoresponsive polymer; and
d. an instruction manual.

The kit was prepared in a manner so that it can be used for the following:
a. processing of whole blood for preparation of PRP of the present disclosure as per example 1;
b. processing of whole blood for preparation of GFC from the PRP of the present disclosure as per example 2;
c. processing of conventional PRP for preparation of GFC of the present disclosure as per example 9;
d. preparing of the therapeutic compositions of the present disclosure comprising GFC and thermosensitive polymer as per example 4; and/or
e. preparing of the therapeutic compositions of the present disclosure comprising GFC and thermosensitive polymer, and PBSCs as per example 5.

In addition to the above 4 components, separate kits were also prepared to comprise one platelet activating agent selected from a group comprising collagen, a calcium salt, hyaluronic acid, and thrombin.

In all these kits, a blood collection container comprising an anti-coagulant was also provided.

All the kits so prepared herein additionally comprise an instruction manual each having steps for: processing of the whole blood for processing of whole blood for preparation of PRP of the present disclosure; processing of whole blood for preparation of GFC from the PRP of the present disclosure; processing of conventional PRP for preparation of GFC of the present disclosure; preparing of the therapeutic compositions of the present disclosure comprising PRP and thermosensitive polymer; and preparing of the therapeutic compositions of the present disclosure comprising GFC and thermosensitive polymer. The instructional manual also comprises steps for processing of PBSCs and inclusion on additional therapeutic agent during preparation of any of the said compositions.

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring 25 back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to anyone of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

1. A platelet-derived growth factor concentrate, wherein the platelet-derived growth factor concentrate is substantially free of platelets, RBCs and WBCs.

2. The platelet-derived growth factor concentrate of claim 1, wherein the growth factors concentrate comprises growth factor(s) selected from a group comprising VEGF, EGF, bFGF, IGF-1, PDGF-BB, TGF-b1 and any combination thereof.

3. The platelet-derived growth factor concentrate of claim 2, wherein concentration of the VEGF ranges from about 500-1300 pg/mL, concentration of the EGF ranges from about 100-2000 pg/mL, concentration of the bFGF ranges from about 25-500 pg/mL, concentration of the IGF-1 ranges from about 500-1000 ng/mL, concentration of the PDGF-BB ranges from about 20-500 ng/mL, and concentration of the TGF-b1 ranges from about 100-2000 ng/mL.

4. The platelet-derived growth factor concentrate of claim 1, wherein the platelet-derived growth factor concentrate is obtained from Platelet Rich Plasma (PRP).

5. The platelet-derived growth factor concentrate of claim 4, wherein
a. the PRP comprises a platelet count that is about 10 to 20-fold greater than starting whole blood sample from same subject,
b. the PRP comprises a red blood cell (RBC) count that is 60 to 90-fold lower than starting whole blood sample from same subject, and/or
c. the PRP comprises a white blood cell (WBC) count that is about 10 to 99-fold lower than starting whole blood sample from same subject.

6. A therapeutic composition comprising the growth factor concentrate of claim 1 and a thermoresponsive polymer.

7. The therapeutic composition of claim 6, wherein the thermoresponsive polymer is selected from a group comprising a copolymer of poly(N-isopropylacrylamide-co-n-butyl methacrylate) and polyethylene glycol, copolymer comprising poly(ethylene oxide) (PEO) and poly(propylene oxide) (PPO), a NIPAM based polymer, amphiphilic block copolymers, ABA triblock copolymers and poloxamer/pluronics family, and any combination thereof.

8. The therapeutic composition of claim 6, wherein concentration of the growth factor ranges from about 10% to 90%; and wherein concentration of the thermoresponsive polymer ranges from about 10% to 50%.

9. The therapeutic composition as claimed of claim 8, wherein the growth factor concentrate and the thermoresponsive polymer are present at a volume/volume ratio of 90:10 to 10:90.

10. The therapeutic composition of claim 6, further comprising peripheral blood stem cells (PBSCs) at a concentration ranging from about 10% to 50%.

11. The therapeutic composition of claim 9, further comprising therapeutic agent selected from the group comprising Vitamin E, human chorionic gonadotropin(HCCG),leukemia inhibitory factor (LIF), Vascular endothelial growth factor (VEGF), Metalloproteinase-9 (MMP-9), Aspirin, Heparin, Sildenafil citrate, estrogen, progesterone, Stem cells, Cells/Stem cell secretome; and wherein the therapeutic composition is fortified with growth factor(s) selected from a group comprising TGF, EGF, 1-IGF-1, bFGF, PDGF, LIF, VEGF, SCF, IL-1b, Fibronectin, IL-1, CSF, HIF-alpha, Activin A,IL-8,TNF-a, NF-kB and any combination thereof.

12. A method for preparing the growth factor concentrate of claim 1, comprising steps of:
a. incubating whole blood with red blood cell (RBC) aggregating agent(s);
b. subjecting the whole blood incubated with the RBC aggregating agent to a first centrifugation to obtain a supernatant containing platelets;
c. subjecting the supernatant to a second centrifugation to obtain a platelet pellet and platelet-poor plasma (PPP);
d. resuspending the platelet pellet in PPP to obtain platelet-rich plasma (PRP);
e. treating the PRP with platelet-activation buffer; and
f. collecting supernatant containing the growth factor concentrate.

13. The method of claim 12, wherein the whole blood is withdrawn from a subject;
and wherein the subject is administered with G-CSF prior to the withdrawal of blood.

14. The method of claim 12, wherein the RBC aggregating agent is selected from a group comprising heparin, collagen, a calcium salt, hyaluronic acid, polygeline, thrombin, gelatin, EDTA, sodium citrate, starch, and any combination thereof; wherein the RBC aggregating agent is added at a concentration of 0.1 to 10% by volume of the whole blood; and
wherein the whole blood is incubated with the RBC aggregating agent for about 5-45 minutes.

15. The method of claim 12, wherein the first centrifugation is carried out at a speed of about 300 rpm to 1000 rpm for about 1-5 minutes; and wherein the second centrifugation is carried out at a speed of about 900 rpm to 4000 rpm for about 10-15 minutes.

16. The method of claim 12, wherein the platelet-activation buffer comprises activating agent(s) selected from a group comprising collagen, a calcium salt, hyaluronic acid, thrombin, and any combination thereof.

17. The method of claim 12, wherein the treatment of step (e) comprises at least one freeze-thaw cycle.

18. A method for preparing the therapeutic composition of claim 6, comprising mixing the platelet derived growth factor concentrate with the thermoresponsive polymer to obtain the composition.

19. The method of claim 18, wherein the platelet derived growth factor concentrate is mixed with the thermoresponsive polymer at a volume/volume ratio of 90:10 to 10:90.

20. The method of claim 19, comprising adding peripheral blood stem cells to the composition.

21-35. (canceled)
